(19) United States
(12) Patent Application Publication
(10) Pub. No.: US 2022/0064311 A1
(43) Pub. Date: Mar. 3, 2022

(54) The use of anti-CCR7 mabs for the prevention or treatment of graft-versus-host disease (GvHD)

(71) Applicants: Catapult Therapeutics B.V., Lelystad (NL); Universidad Autónoma de Madrid, Madrid (ES)

(72) Inventors: Carlos Cuesta Mateos, Madrid (ES); Cecilia Muñoz Calleja, Algete (ES); Itxaso Portero Sainz, Madrid (ES); María del Valle Gómez García de Soria, Madrid (ES); María Luisa Toribio, Madrid (ES); Fernando Terrón Fernández, Madrid (ES)

(73) Assignees: Catapult Therapeutics B.V., Lelystad (NL); Universidad Autónoma de Madrid, Madrid (ES)

(21) Appl. No.: 17/415,004

(22) PCT Filed: Dec. 18, 2019
(86) PCT No.: PCT/EP2019/085991

§ 371 Date: Jun. 17, 2021

(30) Foreign Application Priority Data
Dec. 18, 2018 (EP) 18213727.3
Dec. 11, 2019 (EP) 19215366.6

(51) Int. Cl. C07K 16/28; A61P 37/06

(52) U.S. Cl. CPC C07K 16/2866; A61K 2039/505; C07K 2317/92; A61P 37/06

(57) ABSTRACT

The present invention provides a novel use and methods comprising antibodies, or antigen-binding fragments thereof, which bind to a CCR7 receptor for use as a novel therapeutic agent in prevention and/or treatment of graft versus host disease (GVHD), preferably in hematopoietic stem cell transplantation (HSCT), more preferably allogeneic hematopoietic stem cell transplantation. GVHD of the invention can be acute (aGVHD) and/or chronic (cGVHD), preferably acute. The antibodies and antigen-binding fragments are capable of selectively depleting ex vivo or in vitro immune cells expressing CCR7 and are capable in vivo of selectively killing immune cells expressing a CCR7 receptor and of impairing/blocking migration and of activation of said immune cells, which are involved in the development and evolution of GVHD. The use of said antibodies for depleting, killing and impairing/blocking migration and activation of immune cells expressing CCR7 cells is disclosed, thus providing an alternative therapy for preventing and treating GVHD in both acute and chronic types.

### FIELD OF THE INVENTION

The present invention relates in general to the fields of medicine and pharmacy, in particular to the field of biopharmaceuticals for use in organ, tissue or cell transplantation and grafting. More specifically, the invention relates to anti-CCR7 receptor antibodies that are useful in the prevention and treatment of graft versus host disease.

### BACKGROUND ART

In recent years, hematopoietic stem cell transplantation (HSCT) has been widely performed for the purpose of treating various haematological diseases such as hematopoietic organ tumor, leukaemia, or hypoplastic anaemia. Moreover, cell transplantation is a useful treatment method in the medical field. HSCT is classified, according to differences in the choice of stem cell sources or donors. Common stem cell sources include bone marrow harvested from iliac crests (Aschan. J. Br Med Bull. 2006; 77-78:23-36), granulocyte-colony stimulating factor (G-CSF)- or perixaflor-mobilized peripheral blood stem cells (Bacigalupo et al., Haematologica. 2002 August; 87 (8 Suppl):4-8) and umbilical cord blood (Kestendjieva et al., Cell Biol Int. 2008 July; 32(7):724-32). HSCT can be autologous when the stem cells are derived from the patient itself or allogeneic when the stem cells are from a healthy person, including individual genotypically identical related donors which share major and minor histocompatibility identity, human leukocyte antigen (HLA)-identical sibling donors, HLA-matched donors among extended family members, HLA-identical unrelated donors, mismatched related donors, mismatched unrelated donors, mismatched cord blood donors, and haplotype-mismatched related donors.

However, and despite the use of highly sophisticated therapeutic approaches, HSCT is still associated with a considerable mortality caused by a number of complications such as Graft-versus-Host Disease (GvHD), infectious diseases, veno-occlusive disease, donor graft rejection, and relapses of the underlying diseases, of which GvHD is the most frequent and serious complication after allogenic HSCT that needs to be addressed since it affects up to 30-70% of the patients and is associated with significant morbidity and mortality.

GVHD is classically divided into acute and chronic forms. Acute GVHD (aGVHD) typically occurs between the time of engraftment through 100 days after transplant and chronic GVHD (cGVHD) later than 100 days after HSCT. Both types of GVHD are further subdivided into degrees depending on the clinical severity of the disease. However, this temporal distinction is blurring with the new therapeutic approaches and they have included an overlap syndrome which shares characteristics of both. (Ferrara, J. L., et al., Lancet, 2009. 373(9674): p. 1550-61; Filipovich, A. H., et al., Biol Blood Marrow Transplant, 2005. 11(12): p. 945-56). Furthermore, GVHD is often considered as a single disease, split into two phases: an acute phase of GVHD occurring early after HSCT, and a chronic phase in which GVHD appears later in the course of transplantation (MacDonald et al. Blood. 2017; 129(1):13-21).

Acute GVHD primarily affects the skin, gastrointestinal tract, and liver. Skin lesions usually consist on a maculopapular rash which in the most extreme cases can blister and ulcerate, with bullae and toxic epidermal necrolysis mimicking Stevens-Johnson syndrome. Gastrointestinal manifestations include abdominal cramping and pain, diarrhoea, haematochezia, ileus, anorexia, nausea, and vomiting. Liver disease is due to damage to bile canaliculi which results in cholestasis and therefore hyperbilirubinemia and elevated alkaline phosphatase.

Chronic GVHD usually resembles autoimmune disease like systemic sclerosis with sclerosis and fibrosis usually affecting the skin, eyes, mouth, gut, liver, lungs, joints and genitourinary system. Typical skin manifestations are sclerosis and poikiloderma and lichen-type lesions. In the case of the lung, bronchiolitis obliterans is the result of the damage and obstruction of bronchioles and leads to a high mortality.

The hematopoietic system is also commonly affected in both acute and chronic with thymic damage and cytopenias.

Some methods for preventing, treating or suppressing GVHD have been by the use of immunosuppressant drugs such as calcineurin inhibitors (cyclosporin A and tacrolimus (FK506)), antiproliferative agents (methotrexate and mycophenolate mofetil), mTOR inhibitors (sirolimus or rapamycin) and steroids such as prednisone. Recent approaches are directed to prevent or limit the activation and/or proliferation of autoreactive T or B lymphocytes including the in vivo removal of mature T cells from a transplanted cell population (graft) with cyclophosphamide or anti-thymocyte globulin (ATG), and other treatments like extracorporeal photoapheresis, monoclonal antibodies like rituximab, kinase inhibitors impeding B-cell signalling, expansion of regulatory T cells, etc. However, nevertheless these developments are promising, glucocorticoids still constitute

standard front-line therapy, despite the substantial side effects of long-term use as these agents have a non-specific and a wide immunosuppressive effect, their toxicity is high and thus, infectious diseases due to compromised immune system or recurrence of tumor are becoming a problem (Zeiser and Blazard. N Engl J Med 2017; 377:2565-79. DOI: 10.1056/NEJMra1703472). Therefore, development of an effective treating or preventing method for avoiding GVHD more selectively, and drugs therefore is currently still awaited. There is thus still a need in the art for alternative and improved therapeutic approaches that do not suffer from the severe disadvantages of the prior art methodologies.

Human CC motif receptor 7 (hereinafter referred to as “CCR7”) is a seven transmembrane-spanning domain G-protein coupled receptor (GPCR) that was originally found to be expressed in a lymphocyte-selective manner by EBV infection (Birkenbach et al., 1993, J. Virol. 67: 2209-2220). CCR7 selectively binds two chemokines named CCL19 and CCL21. In homeostasis and inflammation, CCR7 is expressed on naïve T and B lymphocytes, central memory T cells (TCM), some subsets of natural killer cells (NK cells), semimature and mature DCs, and plasmocytoid DCs (Forster R, et al., Cell 1999; 99: 23-33; Comerford I, et al. Cytokine Growth Factor Rev. 2013 June; 24(3):269-83). In these leukocyte subsets CCR7 controls migration, organization, and activation.

Some publications report that donor T cells expressing CCR7 may be associated with pathogenesis in GVDH (Portero et al., 2014, Blood 124:3930; Portero-Sainz et al. 2017, Bone Marrow Transplant. 52, pg: 745-752; Coghill et al., 2010, Blood. 115(23):4914-22). However, none of this literature discloses that targeting of CCR7 can effectively be used for the prevention or treatment of GVHD without the disadvantages of the prior art methodologies side effects.

It is therefore an object of the present invention to provide for medicaments and therapeutic approaches that overcome the disadvantages of the prior art approaches for preventing and treating GVHD. In particular it is an object of the present invention to improve survival rate in allogeneic HSCT.

### SUMMARY OF THE INVENTION

In a first aspect, the invention relates to an anti-CCR7 antibody or antigen-binding fragment thereof, for use in preventing or treating Graft Versus Host Disease (GVHD) in a recipient of a transplant comprising a donor cell. Preferably, the anti-CCR7 antibody has an IC50 of no more than 100 nM for inhibiting at least one of CCR7-dependent intracellular signalling and CCR7 receptor internalization, by at least one CCR7-ligand selected from CCL19 and CCL21. More preferably, the anti-CCR7 antibody inhibits CCR7-dependent intracellular signalling without substantial agonistic effects. Most preferably, the anti-CCR7 antibody has a Kd for the N-terminal extracellular domain of human CCR7 that is not more than a factor 20 higher than the Kd of a reference anti-CCR7 antibody, whereby the reference anti-CCR7 antibody is a mouse anti-CCR7 antibody of which the amino acid sequence of the heavy chain variable domain is SEQ ID NO: 1 and of which the amino acid sequence of the light chain variable domain is SEQ ID NO: 2.

In one embodiment, the anti-CCR7 antibody or antigen-binding fragment thereof, for use in accordance with the invention is a chimeric, humanized or human antibody. Preferably, the anti-CCR7 antibody is an antibody having the HVRs of the anti-human CCR7 antibody of which the amino acid sequence of the heavy chain variable domain is SEQ ID NO: 1 and of which the amino acid sequence of the light chain variable domain is SEQ ID NO: 2.

In one embodiment, the anti-CCR7 antibody or antigen-binding fragment thereof, for use in accordance with the invention, is an anti-CCR7 antibody that effects at least one of killing, inducing apoptosis, blocking migration, blocking activation, blocking proliferation and blocking dissemination of CCR7 expressing cells in the recipient.

In the methods or use of the invention for preventing or treating GVHD in a recipient, the transplant comprising the donor cell preferably is a transplant comprising one or more of an organ, tissue, a progenitor cell, a stem cell and a hematopoietic cell. More preferably, the transplant comprising the donor cell is a transplant comprising a hematopoietic stem or progenitor cell. Most preferably, the recipient suffers from a malignant disorder and wherein preferably, the prevention or treatment of GHVD maintains or promotes the graft versus tumour effect or the graft versus leukaemia effect.

In the methods or use of the invention for preventing or treating GVHD in a recipient, preferably, the prevention or treatment of GHVD comprises at least one of: a) administration of the anti-CCR7 antibody to the recipient prior to that the recipient receives the transplant comprising the donor cell; b) administration of the anti-CCR7 antibody to the recipient after that the recipient has received the transplant comprising the donor cell, and preferably before that the recipient shows symptoms of GHVD or before that the recipient has been diagnosed with GHVD; c) administration of the anti-CCR7 antibody to the recipient after that the recipient has received the transplant comprising the donor cell, and preferably after that the recipient shows symptoms of GHVD or after that the recipient has been diagnosed with GHVD; d) administration of the anti-CCR7 antibody to the recipient of a transplant comprising the donor cell, which transplant has been prepared prior to transplantation by an ex vivo incubation with an anti-CCR7 antibody or antigen-binding fragment as defined above; and, e) administration of the anti-CCR7 antibody to the recipient after recurrence of GHVD.

In a further aspect, the invention pertains to an ex vivo method for preparing an organ, tissue or cell preparation from a donor for transplantation into a recipient, the method comprising the steps of: a) incubating the organ, tissue or cell preparation with an anti-CCR7 antibody or antigen-binding fragment thereof as defined above, whereby the anti-CCR7 antibody effects at least one of: i) a reduction of the number of, and ii) an inhibition of the activity of, CCR7 expressing donor cells in the organ, tissue or cell preparation; and, b) optionally, removal of at least one of the anti-CCR7 antibody and the CCR7 expressing donor cells from the organ, tissue or cell preparation. Preferably, in the method, the anti-CCR7 antibody is comprised in a preservation solution used to preserve the organ, tissue or cell preparation prior to transplantation. More preferably in the method, the organ or tissue is perfused or washed with the preservation solution comprising the anti-CCR7 antibody. Most preferably, in the method, the anti-CCR7 antibody and the CCR7 expressing donor cells are removed from the cell preparation by affinity purification of the anti-CCR7 antibody and the CCR7 expressing donor cells bound thereto.

An ex vivo method according to the invention is preferably used in preparing a transplant to be used in step d) of a method for use according to the invention described above.

### DESCRIPTION OF INVENTION

### Definitions

In the specification, “GVHD” is defined as a disease in which lymphocytes and the like in a graft transplanted into a host recognize host tissues as foreign and attack those tissues. In this case, the term “recipient” or “host” as used herein refers to a subject receiving transplanted or grafted cells, tissue or an organ (transplant patient). These terms may refer to, for example, a subject receiving an administration of donor bone marrow, donor purified hematopoietic progenitors, donor peripheral blood, donor umbilical cord blood, donor T cells, or a pancreatic islet graft. The transplanted tissue may be derived from a syngeneic or allogeneic donor. The term “donor” as used herein refers to a subject from whom tissue is obtained to be transplanted or grafted into a recipient or host. For example, a donor may be a subject from whom bone marrow, peripheral blood, umbilical cord blood, T cells, or other tissue to be administered to a recipient or host is derived. The present invention is mainly targeted at a human and is suitably used for human patients. However, the invention may be used for non-human animals in which at least antibody formation by immune reactions is observed. The term humans identifies any subject as adult subjects and paediatric population, wherein with the term paediatric population is intended the part of population from birth to eighteen (18) years old.

The term “antibody” is used in the broadest sense and specifically covers, e.g. single anti-CCR7 monoclonal antibodies, including antagonist, neutralizing antibodies, full length or intact monoclonal antibodies, anti-CCR7 antibody compositions with polyepitopic specificity, polyclonal antibodies, multivalent antibodies, single chain anti-CCR7 antibodies and fragments of anti-CCR7 antibodies (see below), including Fab, Fab′, F(ab′)2 and Fv fragments, diabodies, single domain antibodies (sdAbs), as long as they exhibit the desired biological and/or immunological activity. The term “immunoglobulin” (Ig) is used interchangeable with antibody herein. An antibody can be human and/or humanized.

The term “anti-CCR7 antibody” or “an antibody that binds to CCR7” refers to an antibody that is capable of binding CCR7 with sufficient affinity such that the antibody is useful as a diagnostic and/or therapeutic agent in targeting CCR7. Preferably, the extent of binding of an anti-CCR7 antibody to an unrelated, non-CCR7 protein is less than about 10% of the binding of the antibody to CCR7 as measured, e.g., by a radioimmunoassay (RIA) or ELISA. In certain embodiments, an antibody that binds to CCR7 has a dissociation constant (K_{d}) of ≤1 mM, ≤100 nM, ≤10 nM, ≤1 nM, or ≤0.1 nM. In certain embodiments, anti-CCR7 antibody binds to an epitope of CCR7 that is conserved among CCR7 from different species.

An “isolated antibody” is one which has been identified and separated and/or recovered from a component of its natural environment.

The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 Daltons. Each L chain is linked to an H chain by one covalent disulphide bond, while the two H chains are linked to each other by one or more disulphide bonds depending on the H chain isotype. Each H and L chain also has regularly spaced intrachain disulphide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, ϵ, ε, γ, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The “variable region” or “variable domain” of an antibody refers to the amino-terminal domains of the heavy or light chain of the antibody. The variable domain of the heavy chain may be referred to as “V_{H}.” The variable domain of the light chain may be referred to as “V_{L}.” These domains are generally the most variable parts of an antibody and contain the antigen-binding sites.

The term “variable” refers to the fact that certain segments of the variable domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” (HVRs) that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), complement dependent cytotoxicity (CDC) and antibody dependent cellular phagocytosis (ADCP).

An “intact” antibody is one which comprises an antigen-binding site as well as a CL and at least heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

A “naked antibody” for the purposes herein is an antibody that is not conjugated to a cytotoxic moiety or radiolabel.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments. In one embodiment, an antibody fragment comprises an antigen binding site of the intact antibody and thus retains the ability to bind antigen.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site, in contrast to polyclonal antibody preparations which include different antibodies directed against different determinants (epitopes). Monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc.), and human constant region sequences.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, a few framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise typically two variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma and Immunol., 1:105-115 (1998); Harris, Biochem. Soc. Transactions, 23:1035-1038 (1995); Hurle and Gross, Curr. Op. Biotech., 5:428-433 (1994).

The term “hypervariable region”, “HVR”, when used herein refers to the regions of an antibody variable domain which are hypervariable in sequence and/or form structurally defined loops that are responsible for antigen binding. Generally, antibodies comprise six hypervariable regions; three in the VH (H1, H2, H3), and three in the VL (L1, L2, L3). A number of hypervariable region delineations are in use and are encompassed herein. The hypervariable regions generally comprise amino acid residues from a “complementarity determining region” or “CDR” (e.g., around about residues 24-34 (L1), 50-56 (L2) and 89-97 (L3) in the VL, and around about 31-35 (H1), 50-65 (H2) and 95-102 (H3) in the VH when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the VL, and 26-32 (H1), 52-56 (H2) and 95-101 (H3) in the VH when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the VL, and 27-38 (H1), 56-65 (H2) and 105-120 (H3) in the VH when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the VL, and 28, 36 (H1), 63, 74-75 (H2) and 123 (H3) in the VH when numbered in accordance with Honneger, A. and Plunkthun, A. J. (Mol. Biol. 309:657-670 (2001)). The hypervariable regions/CDRs of the antibodies of the invention are preferably defined and numbered in accordance with the IMGT numbering system.

“Framework” or “FR” residues are those variable domain residues other than the hypervariable region residues herein defined.

A “blocking” antibody or an “antagonist” antibody is one which inhibits or reduces biological activity of the antigen it binds. Preferred blocking antibodies or antagonist antibodies substantially or completely inhibit the biological activity of the antigen.

An “agonist antibody”, as used herein, is an antibody which mimics at least one of the functional activities of a polypeptide of interest.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{d}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

A “K_{d}” or “K_{d} value” can be measured by using surface plasmon resonance assays using a BIAcore™-2000 or a BIAcore™-3000 (BIAcore, Inc., Piscataway, N.J.) at 25° C. with immobilized antigen CM5 chips at ˜10-50 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 μg/ml (˜0.2 μM) before injection at a flow rate of 5 μl/minute to achieve approximately 10 response units (RU) of coupled protein. Following the injection of antigen, 1M ethanolamine is injected to block unreacted groups. For kinetics measurements, two-fold serial dilutions of the antibody or Fab (0.78 nM to 500 nM) are injected in PBS with 0.05% Tween 20 (PBST) at 25° C. at a flow rate of approximately 25 μl/min. Association rates (kₒₙ) and dissociation rates (k_{off}) are calculated using a simple one-to-one Langmuir binding model (BIAcore Evaluation Software version 3.2) by simultaneous fitting the association and dissociation sensorgram. The equilibrium dissociation constant (K_{d}) is calculated as the ratio k_{off}/kₒₙ. See, e.g., Chen, Y., et al., (1999) J. Mol Biol 293:865-881. If the on-rate exceeds 10⁶ M⁻¹ S⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-Aminco spectrophotometer (ThermoSpectronic) with a stir red cuvette.

An “on-rate” or “rate of association” or “association rate” or “kₒₙ” according to this invention can also be determined with the same surface plasmon resonance technique described above using a BIAcore™-2000 or a BIAcore™-3000 (BIAcore, Inc., Piscataway, N.J.) as described above.

An antibody “which binds” an antigen of interest, e.g. a polypeptide CCR7 antigen target, is one that binds the antigen with sufficient affinity such that the antibody is useful as a therapeutic agent in targeting a cell or tissue expressing the antigen, and does not significantly cross-react with other proteins. In such embodiments, the extent of binding of the antibody to a “non-target” protein will be less than about 10% of the binding of the antibody to its particular target protein as determined by fluorescence activated cell sorting (FACS) analysis or radioimmunoprecipitation (RIA). With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labelled target. In this case, specific binding is indicated if the binding of the labelled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a K_{d} for the target (which may be determined as described above) of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In one embodiment, the term “specific binding” refers to binding where a molecule binds to a particular polypeptide or epitope on a particular polypeptide without substantially binding to any other polypeptide or polypeptide epitope.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity (CDC); Fc receptor binding; antibody-dependent cell-mediated cytotoxicity (ADCC); antibody-dependent cell-mediated phagocytosis (ADCP); down regulation of cell surface receptors (e.g. B cell receptor); and B cell activation.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

A “functional Fc region” possesses an “effector function” of a native sequence Fc region. Exemplary “effector functions” include C1q binding; CDC; Fc receptor binding; ADCC; phagocytosis; down regulation of cell surface receptors (e.g. B cell receptor; BCR), etc. Such effector functions generally require the Fc region to be combined with a binding domain (e.g. an antibody variable domain) and can be assessed using various assays as disclosed, for example, in definitions herein.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. (USA) 95:652-656 (1998). WO 2000/42072 (Presta) describes antibody variants with improved or diminished binding to FcRs. See also, e.g., Shields et al. J. Biol. Chem. 9(2):6591-6604 (2001).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al. (1996, J. Immunol. Methods 202:163), may be performed. Antibody variants with altered Fc region amino acid sequences (antibodies with a variant Fc region) and increased or decreased C1q binding capability are described, e.g. in U.S. Pat. No. 6,194,551 B1 and WO 1999/51642. See also, e.g., Idusogie et al. (2000, J. Immunol. 164: 4178-4184). One such substitution that increases C1q binding, and thereby an increases CDC activity, is the E333A substitution, which can advantageously be applied in the antibodies of the invention.

“Sequence identity” is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. The terms “sequence identity” or “sequence similarity” means that two (poly)-peptide or two nucleotide sequences, when optimally aligned, preferably over the entire length (of at least the shortest sequence in the comparison) and maximizing the number of matches and minimizes the number of gaps such as by the programs ClustalW (1.83), GAP or BESTFIT using default parameters, share at least a certain percentage of sequence identity as defined elsewhere herein. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides, the default scoring matrix used is nwsgapdna and for proteins the default scoring matrix is Blosum62 (Henikoff & Henikoff, 1992, PNAS 89, 915-919). A preferred multiple alignment program for aligning protein sequences of the invention is ClustalW (1.83) using a blosum matrix and default settings (Gap opening penalty:10; Gap extension penalty: 0.05). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have substantially different overall lengths, local alignments, such as those using the Smith Waterman algorithm, are preferred. Alternatively percentage similarity or identity may be determined by searching against public databases, using algorithms such as FASTA, BLAST, etc.

### DETAILED DESCRIPTION OF INVENTION

The invention is based on the finding that CCR7 receptor is highly expressed in some lymphoid cells and antigen-presenting cells (APCs). In said cells, CCR7 plays a main role in the entry into the lymphoid tissues, including lymph nodes (LN), a process underlying development and evolution of GVHD. The present inventors have surprisingly found that an anti-CCR7 antibody produces a remarkable therapeutic effect in GVHD models in mice. GVHD can be suppressed, without noticeable side effects, by administration of an anti-CCR7 antibody to the recipient of the graft. In vivo models show how CCR7 targeting with an antibody prevents disease and ameliorates GVHD once developed, thus, making the CCR7 receptor an interesting target for mAb therapy in both acute and chronic GVHD. Monoclonal antibodies (mAbs) against CCR7, i.e., antibodies which recognize an epitope in a CCR7 receptor and which preferably capable of inhibiting CCR7-dependent intracellular signalling are capable in vivo of killing and/or blocking migration, activation and/or proliferation, and/or dissemination of CCR7⁺ donor and recipient immune cells, whereas they leave CCR7⁻ immune cells unaffected thus maintaining e.g. GVL, and improving GVHD symptoms and survival in vivo.

In a first aspect therefore, the invention relates to anti-CCR7 antibody or antigen-binding fragment thereof, for use in at least one of prevention and treatment of GVHD in a recipient of a transplant comprising a donor cell. Preferably, at least one of the recipient and donor cell are human. The transplant preferably comprises donor cells that comprise an immune cell, more preferably, an immunocompetent cell (e.g., mature T cells) that will cause an immune response against recipient tissues to mediate GVHD. The GVDH can be acute or chronic GVHD. Preferably, the GVDH is acute GVHD. “Treating” GVHD, as used herein, is understood to mean suppressing GVHD, reducing the % occurrence of GVHD, treating GVHD, ameliorating or attenuating one or more clinical manifestations of GVHD, and improving survival rate of treated subjects. “Preventing” GVHD, as used herein, is understood to mean “prophylaxis”. In vivo prophylaxis means suppressing the development of GVHD, delaying the onset of GVHD, reducing the % occurrence of GVHD, reducing the one or more clinical manifestations of GVHD once it occurs, etc.

The anti-CCR7 antibody or antigen-binding fragment thereof, for use in the present invention can be any antigen binding proteins that specifically binds to CCR7. An antigen binding protein of the invention that binds to CCR7 preferably is an anti-CCR7 antibody in the broadest sense as defined herein above, including e.g. anti-CCR7 antibodies, antibody fragments, antibody derivatives, antibody muteins, and antibody variants. An anti-CCR7 antibody of the invention preferably is an isolated antibody. Preferably, an anti-CCR7 antibody of the invention binds to a primate CCR7, more preferably to human CCR7. Reference amino acid sequences of human CCR7 are e.g. NP_001288643, NP_001288645, NP_001288646, NP_001288647, NP_001829, NP_001288642 and NP_031745. Amino acids 1 to 24 of this sequence comprise the membrane translocation signal peptide, which is cleaved off during expression. Amino acids 25 to 59 of human CCR7 make up the N-terminal extracellular domain, which domain comprises sulfated tyrosine residues in position Y₃₂ and Y₄₁. Various allelic variants are known for human CCR7 with one or more amino acid substitutions compared to the above mentioned reference sequences. “Human CCR7” in the present invention includes these allelic variants, at least in as far as the variants have an extracellular domain and the function of CCR7. An anti-CCR7 antibody for use in the invention preferably specifically binds to the N-terminal extracellular domain of a CCR7, preferably a human CCR7.

An anti-CCR7 antibody for use in the invention preferably is a neutralizing antibody that inhibits CCR7-dependent intracellular signalling, CCR7-dependent functions, and/or CCR7 receptor internalization by at least one CCR7 ligand selected from CCL19 and CCL21. An anti-CCR7 antibody preferably has an IC₅₀ that is not higher than 150, 100, 80, 50, 30, 25, 20, 15, 10, 5 or 3 nM for inhibiting CCR7-dependent intracellular signalling and/or CCR7 receptor internalization by at least one CCR7 ligand selected from CCL19 and CCL21, as can e.g. be determined in assay as described in the Examples herein. Alternatively, the maximal IC₅₀ of the antibody is defined by reference to the IC₅₀ of a reference anti-CCR7 antibody when tested in the same assay. Thus, preferably an anti-CCR7 antibody of the invention has an IC₅₀ that is not more than a factor 10, 5, 2, 1.5, 1.2, 1.1 or 1.05 higher than the IC₅₀ of a reference anti-CCR7 antibody, whereby the reference anti-CCR7 antibody is a mouse anti-CCR7 antibody of which the amino acid sequence of the heavy chain variable domain is SEQ ID NO: 1 and of which the amino acid sequence of the light chain variable domain is SEQ ID NO: 2.

An anti-CCR7 antibody of the invention preferably inhibits CCR7-dependent intracellular signalling CCR7 as described above, without substantial agonistic effects, more preferably without detectable agonistic effects, as can e.g. be determined in assay as described in the Examples herein.

An anti-CCR7 antibody for use in the invention preferably has a minimal affinity for the N-terminal extracellular domain of a CCR7, preferably a human CCR7. The minimal affinity of the antibody is herein preferably defined by reference to the K_{d} of a reference anti-CCR7 antibody when tested in the same assay. Thus, preferably an anti-CCR7 antibody of the invention has a K_{d} for the N-terminal extracellular domain of human CCR7 that is not more than a factor 100, 50, 20, 10, 5, 2, 1.5, 1.2, 1.1 or 1.05 higher than the K_{d} of a reference anti-CCR7 antibody for the N-terminal extracellular domain of human CCR7, whereby the reference anti-CCR7 antibody is a mouse anti-CCR7 antibody of which the amino acid sequence of the heavy chain variable domain is SEQ ID NO: 1 and of which the amino acid sequence of the light chain variable domain is SEQ ID NO: 2. It is understood herein that an antibody with a K_{d} that is not more than a factor 10 higher times than the K_{d} of a reference is an antibody that has an affinity that is not less than a factor 10 lower than the affinity of the reference antibody. Thus if the reference antibody has a K_{d} of 1×10⁻⁹ M, the antibody in question has a K_{d} of 1×10⁻⁸ M or less.

Examples of anti-CCR7 antibodies with one or more of the above-defined characteristics and suitable for use in the present invention include e.g. the monoclonal antibodies described in U.S. Pat. No. 8,865,170, WO 2009/139853, WO 2014/151834 and WO 2017/025569, all of which are incorporated herein by reference.

A preferred anti-CCR7 antibody for use in the present invention is an antibody that specifically binds to an epitope comprising or consisting of the amino acid sequence “ZxLFE”, wherein Z is a sulfated tyrosine and x can be any amino acid and F can be replaced by a hydrophobic amino acid. The antibody of the invention thus preferably specifically binds to an epitope comprising or consisting of the amino acids sequence “ZTLFE” in positions 41 to 45 in the N-terminal extracellular domain of human CCR7. The antibody preferably is specific for human CCR7. Such a preferred anti-CCR7 antibody preferably has a minimal affinity for human CCR7 or for a synthetic antigen comprising the “ZTLFE” epitope, preferably for the synthetic antigen SYM1899 as described in the Examples herein. Preferably therefore, the anti-CCR7 antibody has a K_{d} of 1×10⁻⁸ M, 5×10⁻⁹ M, 2×10⁻⁹ M, 1.8×10⁻⁹ M, 1×10⁻⁹ M, 1×10⁻¹⁰ M or 1×10⁻¹¹ M or less preferably for the synthetic antigen SYM1899. Alternatively, the minimal affinity of the antibody is defined by reference to the K_{d} of a reference anti-CCR7 antibody when tested in the same assay. Thus, preferably an anti-CCR7 antibody of the invention has a K_{d} for human CCR7 or for a synthetic antigen comprising the “ZTLFE” epitope (preferably the synthetic antigen SYM1899 as described in the Examples herein) that is not more than a factor 10, 5, 2, 1.5, 1.2, 1.1 or 1.05 higher than the K_{d} of a reference anti-CCR7 antibody for the antigen, whereby the reference anti-CCR7 antibody is a mouse anti-CCR7 antibody of which the amino acid sequence of the heavy chain variable domain is SEQ ID NO: 1 and of which the amino acid sequence of the light chain variable domain is SEQ ID NO: 2. It is understood herein that an antibody with a K_{d} that is not more than a factor 10 higher times than the K_{d} of a reference is an antibody that has an affinity that is not less than a factor 10 lower than the affinity of the reference antibody. Thus if the reference antibody has a K_{d} of 1×10⁻⁹ M, the antibody in question has a K_{d} of 1×10⁻⁸ M or less.

An anti-CCR7 antibody for use in the invention preferably binds to human CCR7 or to a synthetic antigen comprising the “ZTLFE” epitope (preferably the synthetic antigen SYM1899 as described in the Examples herein; SEQ ID NO: 3) with a maximal k_{off} rate constant. Preferably therefore, the anti-CCR7 antibody of the invention has a k_{off} rate constant that is 1×10⁻³, 1×10⁻⁴ or 1×10⁻⁵ s⁻¹ or less. Alternatively, the maximal k_{off} rate constant of the antibody is defined by reference to the k_{off} rate constant of a reference anti-CCR7 antibody when tested in the same assay. Thus, preferably an anti-CCR7 antibody of the invention binds to human CCR7 or to a synthetic antigen comprising the “ZTLFE” epitope (preferably the synthetic antigen SYM1899 as described in the Examples herein) that is not more than a factor 10, 5, 2, 1.5, 1.2, 1.1 or 1.05 higher than the k_{off} rate constant of a reference anti-CCR7 antibody for the antigen, whereby the reference anti-CCR7 antibody is a mouse anti-CCR7 antibody of which the amino acid sequence of the heavy chain variable domain is SEQ ID NO: 1 and of which the amino acid sequence of the light chain variable domain is SEQ ID NO: 2.

One such preferred antibody for use in the present invention is an antibody having the HVRs of the reference mouse anti-human CCR7 antibody of which the amino acid sequence of the heavy chain variable domain is SEQ ID NO: 1 and of which the amino acid sequence of the light chain variable domain is SEQ ID NO: 2, which HVRs are defined in WO 2017/025569, incorporated by reference herein.

An anti-CCR7 antibody for use in the invention can be a chimeric antibody, e.g. mouse-human antibody. However, preferably the antibody is a humanized or human antibody.

A humanized antibody for use in the invention preferably elicits little to no immunogenic response against the antibody in a subject to which the antibody is administered. For example, a humanized antibody for use in the invention elicits and/or is expected to elicit a human anti-mouse antibody response (HAMA) at a substantially reduced level compared to the original mouse an antibody, e.g. comprising the sequence of SEQ ID NO: 1 and 2 in a host subject. Preferably, the humanized antibody elicits and/or is expected to elicit a minimal or no human anti-mouse antibody response (HAMA). Most preferably, an antibody of the invention elicits anti-mouse antibody response that is at or less than a clinically-acceptable level.

Humanization can be essentially performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting hypervariable region sequences for the corresponding sequences of a human antibody. In practice, humanized antibodies are typically human antibodies in which some hypervariable region residues and possibly some framework region (FR) residues are substituted by residues from analogous sites in rodent antibodies. The choice of human variable domains, both light and heavy, to be used in making the humanized antibodies is very important to reduce immunogenicity retaining the specificity and affinity for the antigen. According to the so called “best-fit” method, the sequence of the variable domain of a rodent antibody is screened against the entire library of known human variable-domain sequences. The human sequence which is closest to that of the rodent is then accepted as the human framework region (FR) for the humanized antibody (Suns et al., J. Immunol., 151:2296 (1993); Chothia et al., J. Mol. Biol, 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies are humanized, with retention of high affinity for the antigen and other favourable biological properties. To achieve this goal, according to a preferred method, humanized antibodies are prepared by a process of analysis of the parental sequences and various conceptual humanized products using three-dimensional models of the parental and humanized sequences. A humanized anti-CCR7 antibody, according to any of the above embodiments of the invention, preferably comprises a heavy chain constant region that is IgG1, IgG2, IgG3 or IgG4 region. A humanized anti-CCR7 antibody according to any of the above embodiments of the invention, preferably comprises a functional Fc region possessing at least one effector function selected from the group consisting of: C1q binding, complement dependent cytotoxicity; Fc region binding, antibody-dependent cell-mediated cytotoxicity and phagocytosis.

A preferred humanized antibody for use in the present invention is an antibody of which the amino acid sequence of the heavy chain variable domain is SEQ ID NO: 4 and of which the amino acid sequence of the light chain variable domain is SEQ ID NO: 5, as e.g. described in WO 2017/025569.

As an alternative to humanization, human antibodies can be generated. By “human antibody” is meant an antibody containing entirely human light and heavy chains as well as constant regions, produced by any of the known standard methods. For example, transgenic animals (e.g., mice) are available that are capable, upon immunization, of producing a full repertoire of human antibodies in the absence of endogenous immunoglobulin production. For example, it has been described that the homozygous deletion of the antibody heavy-chain joining region PH gene in chimeric and germ-line mutant mice results in the complete inhibition of endogenous antibody production. Transfer of the human germ-line immunoglobulin gene array in such germ line mutant mice will result in the production of human antibodies after immunization. See, e.g., Jakobovits et al., Proc. Nat. Acad. Sci. USA, 90:255 1 (1993); Jakobovits et al., Nature, 362:255-258 (1993). Alternatively, phage display technology (McCafferty et al., Nature 348:552-553 (1990)) can be used to produce human antibodies and antibody fragments in vitro, from immunoglobulin variable (V) domain gene repertoires from donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-57 1 (1993). Human antibodies may also be generated by in vitro activated B cells or SCID mice with its immune system reconstituted with human cells. Once a human antibody is obtained, its coding DNA sequences can be isolated, cloned and introduced into an appropriate expression system i.e. a cell line, preferably from a mammal, which subsequently express and liberate it into a culture media from which the antibody can be isolated.

A preferred human antibody for use in the present invention is an antibody of which the amino acid sequence of the heavy chain variable domain is SEQ ID NO: 6 and of which the amino acid sequence of the light chain variable domain is SEQ ID NO: 7 or 8, as e.g. described in WO 2014/151834.

Functional fragments of antibodies which bind to a CCR7 receptor that are included for use within the present invention retain at least one binding function and/or modulation function of the full-length antibody from which they are derived. Preferred functional fragments retain an antigen-binding function of a corresponding full-length antibody (e.g., the ability to bind a mammalian CCR7 receptor). Particularly preferred functional fragments retain the ability to inhibit one or more functions characteristic of a mammalian CCR7 receptor, such as a binding activity and/or blocking a signalling activity, and/or stimulation of a cellular response. For example, in one embodiment, a functional fragment can inhibit the interaction of CCR7 with one or more of its ligands and/or can inhibit one or more receptor-mediated functions.

In some embodiments, an anti-CCR7 antibody of the invention comprises a light chain and/or a heavy chain antibody constant region. Any antibody constant regions known in the art can be used. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. An anti-CCR7 antibody of the invention can thus have constant regions of any isotype, i.e. including IgG, IgM, IgA, IgD, and IgE constant regions as well as IgG1, IgG2, IgG3, or IgG4 constant regions. In one embodiment, the light or heavy chain constant region is a fragment, derivative, variant, or mutein of a naturally occurring constant region. Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See also Lantto et al. (2002, Methods Mol. Bio1. 178:303-16). Accordingly, the anti-CCR7 antibodies of the invention include those comprising, for example, one or more of the variable domain sequences disclosed herein and having a desired isotype (e.g., IgA, IgGI, IgG2, IgG3, IgG4, IgM, IgE, and IgD), as well as Fab or F(ab′)2 fragments thereof. Moreover, if an IgG4 is desired, it may also be desired to introduce a point mutation (CPSCP->CPPCP) in the hinge region as described in Bloom et al. (1997, Protein Science 6:407) to alleviate a tendency to form intra-H chain disulfide bonds that can lead to heterogeneity in the IgG4 antibodies.

An anti-CCR7 antibody of the invention preferably comprises a functional Fc region possessing at least one effector function selected from the group consisting of: C1q binding, complement dependent cytotoxicity; Fc receptor binding, antibody-dependent cell-mediated cytotoxicity and phagocytosis.

An anti-CCR7 antibody of the invention can be modified to improve effector function, e.g. so as to enhance ADCC and/or CDC of the antibody. This can be achieved by introducing one or more amino acid substitutions in an Fc region of an antibody. A preferred substitution in the Fc region of an antibody of the invention is a substitution that increases C1q binding, and thereby an increases CDC activity, such as e.g. described in Idusogie et al. (2000, J. Immunol. 164: 4178-4184). A preferred substitution in the Fc region that increases C1q binding is the E333A substitution.

Glycosyl groups added to the amino acid backbone of glycoproteins e.g. antibodies are formed by several monosaccharides or monosaccharide derivatives in resulting in a composition which can be different in the same antibody produced in cell from different mammals or tissues. In addition, it has been shown that different composition of glycosyl groups can affect the potency in mediating antigen-dependent cell-mediated cytotoxicity (ADCC) and/or complement dependent cytotoxicity (CDC) of the antibody. Therefore it is possible to improve those properties by mean of studying the pattern of glycosylation of antibodies from different sources. An example of such approach is Niwa et al. (2004, Cancer Res, 64(6):2127-33).

Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al. (1992, J. Exp Med. 176:1191-1195) and Shopes, (1992, Immunol. 148:2918-2922). Homodimeric antibodies with enhanced anti-tumour activity may also be prepared using heterobifunctional cross-linkers as described in Wolff et al. (1993, Cancer Research 53:2560-2565). Alternatively, an antibody which has dual Fc regions can be engineered and may thereby have enhanced complement lysis and ADCC capabilities. See Stevenson et al. (1989, Anti-Cancer Drug Design 3:2 19-230). In order to increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

A preferred anti-CCR7 antibody of the invention comprises a heavy chain constant region of the human allotype G1m17,1 (see Jefferis and Lefranc (2009) MAbs Vol. 1 Issue 4, pp 1-7), which heavy chain constant region comprises the amino acid sequence of SEQ ID NO: 9. More preferably, the heavy chain constant region of the human allotype G1m17,1 in the antibody of the invention comprises an E333A substitution, which heavy chain constant region comprises the amino acid sequence of SEQ ID NO: 10.

Anti-CCR7 antibodies for use in the invention can be prepared by any of a number of conventional techniques. They will usually be produced in recombinant expression systems, using any technique known in the art. See e.g. Shukla and Thömmes (2010, “Recent advances in large-scale production of monoclonal antibodies and related proteins”, Trends in Biotechnol. 28(5):253-261), Harlow and Lane (1988) “Antibodies: A Laboratory Manual”, Cold Spring Harbor Laboratory Press, Cold Spring Harbor, N.Y., and Sambrook and Russell (2001) “Molecular Cloning: A Laboratory Manual (3^{rd} edition), Cold Spring Harbor Laboratory, Cold Spring Harbor Laboratory Press, NY. Any expression system known in the art can be used to make the recombinant polypeptides of the invention. In general, host cells are transformed with a recombinant expression vector that comprises DNA encoding a desired polypeptide.

In one embodiment, the invention relates to the use of an anti-CCR7 antibody as herein defined above, or antigen-binding fragment thereof, for treating and/or preventing GVHD in a recipient of a transplant comprising donor cells, wherein preferably, the anti-CCR7 antibody effects include at least one of killing, induction of apoptosis, blocking of migration and/or blocking of dissemination of CCR7 expressing cells, blocking of activation of CCR7 expressing cells, blocking of maturation and differentiation of CCR7 expressing cells, preferably in the recipient. The CCR7 expressing cells on which the anti-CCR7 antibody exerts one or more of these effects are preferably CCR7 expressing immune cells, which can be transplanted immune cells derived from the donor or can be host derived immune cells, i.e. derived from the recipient. Examples of donor- or host-derived CCR7 expressing immune cells include e.g. T cells both CD4+ and CD8+ T cells, such as e.g. naïve T cells, central memory T cells, regulatory T cells, helper T cells and cytotoxic T cells, B cells, such as e.g. naïve B cells and follicular B cells, antigen-presenting cells (APC), such as e.g. dendritic cells including e.g. mature dendritic cells (mDC) and plasmacytoid DC. Such as cells expressing a CCR7 receptor can be identified by conventional methods; for example, surface expression of CCR7 receptor can be analyzed by flow cytometry as is generally known in the art. Death of the cells expressing a CCR7 receptor can be determined by any conventional method, for example, by determining absence or clearance of CCR7⁺ cells from the recipient. Preferably, use of an anti-CCR7 antibody in accordance with the invention prevents or reduces infiltration of CD45⁺ donor cells in at least one of a lymph node, peripheral blood, spleen, thymus and bone marrow, among others lymphoid organs of the recipient or into any of the epithelial target tissues of GVHD in the recipient, more preferably, the anti-CCR7 antibody or antigen-binding fragment thereof prevents or reduces infiltration of CCR7⁺, CD45⁺ donor cells in at least one of a lymph node, peripheral blood, spleen, thymus and bone marrow in the recipient, among others lymphoid organs of the recipient or into any of the epithelial target tissues of GVHD in the recipient.

Without wishing to be bound by theory, the therapeutic use of an anti-CCR7 antibody in accordance with the invention advantageously should allow specific prevention or treatment of GVHD in vivo by, e.g. killing CCR7⁺ T cells and APCs, and/or by impairing migration and/or blocking dissemination of CCR7⁺ T cells and APCs, and/or by impairing or blocking activation or differentiation or maturation of CCR7+ T cells and APCs in the recipient. In most cases, complement-dependent cell lysis (CDC), antibody-dependent cell-mediated phagocytosis (ADCP), and antibody-dependent cell-mediated cytotoxicity (ADCC) are believed to be responsible for the clinical utility of the unconjugated anti-CCR7 antibody, although the induction of apoptosis or cell cycle arrest could also play a substantial role. In the case of the application of anti-CCR7 antibodies, impairing and/or blocking migration and/or impairing or blocking activation, differentiation, proliferation or maturation of immune cells is an additional relevant mechanism of action.

Preferably therefore, in one embodiment, the invention relates to use of an anti-CCR7 antibody in accordance with the invention wherein the anti-CCR7 antibody impairs migration of donor and/or recipient cells expressing a CCR7 receptor to secondary lymphoid tissue and/or for blocking dissemination of donor cells into secondary lymphoid tissues including lymph nodes, spleen, and mucose-associated lymphoid tissues (MALT) such as Peyer patches.

The recipient of the transplant in whom GVHD is prevented or treated in accordance with the invention, preferably, is a recipient of a transplant or graft comprising an organ, a progenitor cell, a stem cell, a hematopoietic cell, a hematopoietic progenitor cell or a hematopoietic stem cell. The transplant or graft can be a syngeneic or an allogeneic transplant but preferably is a transplant or graft comprising allogeneic donor cells. The transplant can comprise any type of organ or tissue, including e.g. heart, lung, kidney, liver, pancreas, intestine, face (or parts thereof), cornea, skin, hand, leg, penis, bone, uterus, thymus, etc.

In a preferred embodiment, the anti-CCR7 antibody or the antigen-binding fragment of the invention is used to prevent or treat GVHD in a recipient of a hematopoietic cell graft. More specifically, to prevent or treat GVHD after allogeneic hematopoietic stem cell transplantation (HSCT).

The donor cells used in the methods of the invention may be whole or purified bone marrow cells, purified hematopoietic progenitors or stem cells from the bone marrow, purified hematopoietic progenitor cells or stem cells from the peripheral blood, (purified) umbilical cord blood cells or peripheral blood cells from an apheresis product enriched in hematopoietic progenitors or stem cells after mobilizing hematopoietic progenitors from the bone marrow with growth factors like G-CSF or anti-CXCR4 agents such as plerixafor. In methods of the invention where donor T cells are introduced, the cell graft may comprise whole or purified bone marrow cells, umbilical cord blood cells, or purified stem cells with an add-back of T-cells. Thus, in one embodiment, the donor cells to be used in accordance with the invention comprise, or are derived from, at least one of: T cells, spleen, umbilical cord blood, amniotic fluid, and dental pulp cells from Wharton's jelly, placenta-derived cells, hair-root-derived cells, and/or fat-tissue-derived cells, a cell suspension comprising lymphocytes, monocytes and/or macrophages, a stem-cell-containing tissue, a stem-cell-containing organ, an immune cell containing tissue, and an immune cell containing organ. In one embodiment, the donor cells to be used in accordance with the invention are hematopoietic stem cells (also known as hematopoietic progenitor cells) that comprise, or are derived from bone marrow stem cells, peripheral blood stem cells, umbilical cord blood stem cells, adult stem cells of the bone marrow such as non-adherent bone marrow derived cells (NA-BMCs), embryonic stem cells and/or reprogrammed adult stem cells (i.e. induced pluripotent cells).

The recipient of the hematopoietic (stem) cell graft can have a hematologic disorder or a non-hematologic disorder. The hematologic disorder can be a non-neoplastic hematologic disorder or hematologic malignancy. The non-malignant hematologic disorder, particularly a hematopoietic cell deficiency disorder can be selected from the group consisting of: a congenital or acquired immune deficiency, a genetic disorder causing hemoglobinopathy, an enzyme deficiency disease, or an autoimmune disease, severe aplastic anemia, thalassemia, sickle cell anemia, immunological defects, severe combined immunodeficiency (SCID), Wiskott-Aldrich syndrome (WAS), hemophagocytic lymphohistiocytosis (HLH), inborn errors of metabolism, lysosomal storage disorders, disorders of peroxisomal function, autoimmune diseases, rheumatologic diseases, and recidivisms of any of the above. The hematologic malignancy can be selected from the group consisting of: leukemia, acute myeloid leukemia (AML), promyelocytic leukemia (PML), acute lymphoblastic leukemia (ALL), chronic lymphocytic leukemia (CLL), mantle cell lymphoma (MCL), chronic myelogenous leukemia (CML), myelodysplastic syndrome (MDS), non-Hodgkin's lymphoma (NHL), Hodgkin's lymphoma (HL), multiple myeloma (MM), and neuroblastoma. The recipient of the hematopoietic (stem) cell graft can have non hematological solid tumors (eg renal cell cancer, colorectal cancer, etc).

The recipient of the hematopoietic (stem) cell graft can or cannot have been treated in a myeloablative conditioning regimen, or in a non-myeloablative conditioning regimen or in an reduced-intensity conditioning, preferably prior to receiving the hematopoietic (stem) cell graft.

In one embodiment, the invention relates to use of an anti-CCR7 antibody in accordance with the invention wherein the prevention or treatment of GHVD comprises administration of the anti-CCR7 antibody to the recipient before, at (about) the same time and/or after that the recipient receives the transplant comprising the donor cell. When the anti-CCR7 antibody is administered at (about) the same time that the recipient receives the transplant comprising the donor cell preferably means that the anti-CCR7 antibody is administered within 96, 72, 24, 12, 6 or 3 hours from each other. Preferably, the prevention or treatment of GHVD comprises at least one of: a) administration of the anti-CCR7 antibody to the recipient prior to that the recipient receives the transplant comprising the donor cell; b) administration of the anti-CCR7 antibody to the recipient 48, 72 or 96 hours after that the recipient receives the transplant comprising the donor cell, c) administration of the anti-CCR7 antibody to the recipient after that the recipient receives the transplant comprising the donor cell, and preferably after that the recipient shows symptoms of GHVD or after that GHVD has been diagnosed in the recipient; and, d) administration of the anti-CCR7 antibody to the recipient after recurrence of GHVD.

Administration of the anti-CCR7 antibody prior to that the recipient receives the transplant is believed desirable in that it will condition the recipient for the receipt of the transplant comprising the donor cells and may thus allow to prevent GHVD or at least reduce the risk of GHVD to occur. Preferably, therefore the anti-CCR7 antibody is administered at least prior to that the recipient receives the transplant, more preferably at least 5, 10, 20 or 40 minutes or 1, 2, 4, 8, 12, 24 or 48 hours prior to that the recipient receives the transplant.

Administration of the anti-CCR7 antibody after that the recipient receives the transplant is believed to be desirable in that it will reduce donor-immune attack on the recipient host and further promote acceptance by the recipient of the donor's transplant and/or cells. Preferably, the anti-CCR7 antibody is administered after that the recipient receives the transplant, as long and as often as necessary to reduce the occurrence of GVHD and/or to ameliorate or attenuate one or more symptoms of GVHD. The frequency and dosing of administration will also depend on the serum half-life of the anti-CCR7 antibody and may be adapted accordingly. In a preferred embodiment of the invention the anti-CCR7 antibody is administered both before and after that the recipient receives the transplant.

However, as the Examples herein demonstrate, administration of the anti-CCR7 antibody to the recipient after that allo-reactive responses had developed in recipients having received a transplant, is still effective in treating GHVD, at least in terms of improving survival rate. In one embodiment of the invention therefore, the anti-CCR7 antibody is administered to a recipient having a transplant comprising donor cells, after that the recipient shows clinical manifestations of GHVD and/or detectable allo-reactive responses, and/or preferably after that GHVD has been diagnosed in the recipient. In such instances the recipient may not have received prior treatment with or administration(s) of an anti-CCR7 antibody.

Anti-CCR7 antibody that are administered to the recipient after the recipient has received the transplant can be administered at least 1, 2, 3, 5, 7, 10, 14, 21 or 28 days after at least one of: i) receipt of the transplant or graft by the recipient; ii) the occurrence of symptoms of GHVD in the recipient; iii) detection of an allo-reactive response in the recipient; and, iv) the recipient having been diagnosed with GHVD.

In another embodiment of the invention, the anti-CCR7 antibody is administered to a recipient of a transplant comprising the donor cell, which transplant has been prepared prior to transplantation by an ex vivo incubation with the anti-CCR7 antibody, preferably in accordance with a method as described below. The anti-CCR7 antibody that is administered to the recipient can but need not be the same as the anti-CCR7 antibody that is used in the ex vivo method for preparing the transplant prior to transplantation.

In a preferred embodiment of the invention, the anti-CCR7 antibody or antigen-binding fragment thereof is administered at least once separate from the transplant, preferably shortly before or shortly after administering the transplant. With “shortly” in this context is meant within 24 hours, preferably within 8 hours, more preferably within 6 hours, more preferably within 4 hours, more preferably within 2 hours, most preferably within 1 hour. With “separate from” is meant that the administration of the CCR7 antibody or antigen-binding fragment thereof is comprised in another container, e.g., a syringe, than the transplant. Preferably the CCR7 antibody or antigen-binding fragment thereof is administered at least 10 seconds, more preferably at least one minute prior to, more preferably at least 10 minutes prior to, most preferably at least 1 hour prior to the administration of the transplant. In another preferred embodiment, the transplant is administered at least 10 seconds, more preferably at least one minute prior to, more preferably at least 10 minutes prior to, most preferably at least 1 hour prior to the administration of the CCR7 antibody or antigen-binding fragment thereof.

Preferably, the treatment thus comprises at least one administration to the recipient of the anti-CCR7 antibody or antigen-binding fragment thereof separate from the transplant.

In yet another embodiment of the invention, the anti-CCR7 antibody is administered to a recipient after recurrence of GHVD, whereby the recipient may not have received prior treatment with or administration(s) of an anti-CCR7 antibody.

In another aspect, the invention relates to a pharmaceutical composition comprising an anti-CCR7 antibody (or antigen-binding fragment thereof) as herein defined, for a use in accordance with the invention. The pharmaceutical composition preferably at least comprises the anti-CCR7 antibody or a pharmaceutically derivative or prodrug thereof, together with a pharmaceutically acceptable carrier, adjuvant, or vehicle, for administration to a subject. Said pharmaceutical composition can be used in the methods of treatment described herein below by administration of an effective amount of the composition to a subject in need thereof. The term “subject” is used interchangeably with the term “recipient” herein, and as used herein, refers to all animals classified as mammals and includes, but is not restricted to, primates and humans. The subject is preferably a male or female human of any age or race.

The term “pharmaceutically acceptable carrier”, as used herein, is intended to include any and all solvents, dispersion media, coatings, antibacterial and antifungal agents, isotonic and absorption delaying agents, and the like, compatible with pharmaceutical administration (see e.g. “Handbook of Pharmaceutical Excipients”, Rowe et al eds. 7th edition, 2012, www.pharmpress.com). The use of such media and agents for pharmaceutically active substances is well known in the art. Except insofar as any conventional media or agent is incompatible with the active compound, use thereof in the compositions is contemplated. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or non-ionic surfactants such as TWEEN™, PLURONICS™ or polyethylene glycol (PEG).

The antibodies of the invention may be in the same formulation or may be administered in different formulations. Administration can be concurrent or sequential, and may be effective in either order.

Supplementary active compounds can also be incorporated into the pharmaceutical composition of the invention. Thus, in a particular embodiment, the pharmaceutical composition of the invention may also contain more than one active compound as necessary for the particular indication being treated, preferably those with complementary activities that do not adversely affect each other. For example, it may be desirable to further provide a chemotherapeutic agent, a cytokine, an analgesic agent, or an immunomodulating agent, e.g. an immunosuppressive agent or an immunostimulating agent. The effective amount of such other active agents depends, among other things, on the amount of antibody of the invention present in the pharmaceutical composition, the type of disease or disorder or treatment, etc.

Besides use in a single-agent treatment or prevention of GVHD, the antibodies and pharmaceutical compositions of this invention may be used with other drugs to provide a combination therapy. The other drugs may form part of the same composition, or be provided as a separate composition for administration at the same time or at different time. The combination therapy may have synergistic therapeutic effects on the patients. In a particular embodiment, the antibody of the invention may be combined with other treatments of the medical conditions described herein. The other therapeutic agents include, but are not limited to alkylating agents (e.g., nitrogen mustards, [such as mechloretamine], cyclophosphamide, melphalan and chloambucil), alkyl sulphonates (e.g. busulphan), nitrosoureas (e.g., carmustine, lomustine, semustine and streptoxocine), triazenes (e.g., dacarbazine), antimetabolites (e.g., folic acid analogs such as methotrexate), pyrimidine analogs (e.g., fluorouracil and cytarabine), purine analogs (e.g., fludarabine, idarubicin, cytosine arabinoside, mercaptopurine and thioguanine), vinca alkaloids (e.g., vinblastine, vincristine and vendesine), epidophyllotoxins (etoposide and teniposide), antibiotics (dactinomycin, daunorubicin, doxorubicin, bleomycin, plicamycin and mitomycin), dibromomannitol, deoxyspergualin, dimethyl myleran and thiotepa, proteasomal inhibitors (bortezomib), Pentostatin, immunosuppressant agents such as steroids (e.g., prednisone and methylprednisolone), calcineurin inhibitors (e.g. cyclosporin A, tacrolimus or FK506), mammalian target of rapamycin (mTOR) inhibitors (sirolimus or rapamycin), mycophenolate mofetil, thalidomide, lenalidomide, azathioprine, monoclonal antibodies (e.g., Daclizumab (anti-interleukin (IL)-2), Infliximab (anti-tumor necrosis factor), etanercept, MEDI-205 (anti-CD2), abx-cbl (anti-CD147)), alemtuzumab (anti-CD52), rituximab (anti-CD20), and polyclonal antibodies (e.g., ATG (anti-thymocyte globulin), antihistamines, chemotherapy, radiation therapy, immunotherapy, surgery, alkylating agents, antimetabolites, antihormones, therapeutic for various symptoms, e.g., painkillers, diuretics, antidiuretics, antivirals, antibiotics, cytokines, nutritional supplements, anemia therapeutics, blood clotting therapeutics, bone therapeutics, psychiatric and psychological therapeutics, and the like. In addition, the antibodies and pharmaceutical compositions of this invention may be used in conjunction with other types of therapy as prophylaxis for GVHD prior or at about the same time of transplantation, including but not limited to immunosuppressant agents such as calcineurin inhibitors (e.g. cyclosporin A, tacrolimus or FK506), mammalian target of rapamycin (mTOR) inhibitors (sirolimus or rapamycin), or antiproliferative agents (e.g. mycophenolate mofetil, methotrexate), thymic irradiation, phototherapy, melphalan, in vivo depletion of T cells with cyclophosphamide or ATG, or ex vivo depletion of T cells with antibodies (e.g. anti-CD3) to prevent the onset of GVHD. In addition, the antibodies and pharmaceutical compositions of the invention may be used in conjunction with other types of therapy as treatment for GVHD including but not limited to steroids (e.g., prednisone and methylprednisolone), extracorporeal photopheresis, pentostatin, kinase inhibitors (e.g. ruloxitinib, ibrutinib), proteasoma inhibitors (bortezomib), cellular therapy with NK cells or regulatory T cells or mesenchymal stem cells, immunotherapy with monoclonal antibodies (e.g. rituximab, alemtuzumab, tocilizumab, etc), or fusion proteins (e.g. abatacept, alefacept), inhibitors of the T cell migration (e.g. maraviroc), etc.

It may also be useful to treat patients with cytokines in order to up-regulate the expression of CCR7 or other target protein on the surface of target cells prior to administration of an antibody of the invention. Cytokines may also be administered simultaneously with or prior to or subsequent to administration of the depleting antibody or radiolabeled antibody in order to stimulate immune effector functions.

In addition, the use of anti-CCR7 antibodies for the treatment or prevention of GVHD in accordance with this invention may further include the administration of conditioning regimens to the recipient of the transplant including myeloablative, non-myeloablative or reduced-intensity conditioning treatments prior to the transplant. These treatments eradicate the underlying disease and suppresses and eradicate the host immune system which allow donor stem cells to home into the bone marrow without the risk of graft rejection. The administration of myeloablative or reduced-intensity or non-myeloablative treatments may be used to induce mixed hematopoietic chimerism or full hematopoietic chimerism. Total body irradiation (TBI) and/or chemotherapy regimens with busulfan and/or cyclophosphamide are examples of myeloablative regimens. As used herein, “non-myeloablative” refers to a treatment which kills marrow cells but will not, in a significant number of recipients, lead to death from marrow failure. This allows donor stem cells engraft al least with mixed donor/recipient chimerism. The final elimination of host hematopoiesis is achieved by graft versus host effects of the immune donor cells, which eventually results in full donor chimerism. Low dose TBI, fludarabine, ATG, reduced doses of busulfan or combinations of those are used as non-myeloablative regimens. RIC regimen is an intermediate approach which prevents the high toxicity of myeloablative regimens but provide enough control of the underlying disease and enough immune suppression to prevent graft rejection. A common RIC regimen includes fludarabine and melphalan, but many other agents have been introduced for RIC treatments.

In an embodiment, the antibody of the invention is prepared with carriers that will protect said compound against rapid elimination from the body, such as a controlled release formulation, including implants and microencapsulated delivery systems, e.g. liposomes. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions, including targeted liposomes can also be used as pharmaceutically acceptable carriers. These can be prepared according to methods known to those skilled in the art, for example, as described in U.S. Pat. No. 4,522,811, WO2010/095940.

The administration route of the antibody (or fragment thereof) of the invention can be oral, parenteral, by inhalation or topical. The term “parenteral” as used herein includes intravenous, intra-arterial, intralymphatic, intraperitoneal, intramuscular, subcutaneous, rectal or vaginal administration. The intravenous forms of parenteral administration are preferred. By “systemic administration” is meant oral, intravenous, intraperitoneal and intramuscular administration. The amount of an antibody required for therapeutic or prophylactic effect will, of course, vary with the antibody chosen, the nature and severity of the condition being treated and the patient. In addition, the antibody may suitably be administered by pulse infusion, e.g., with declining doses of the antibody. Preferably the dosing is given by injections, most preferably intravenous or subcutaneous injections, depending in part on whether the administration is brief or chronic.

Thus, in a particular embodiment, the pharmaceutical composition of the invention may be in a form suitable for parenteral administration, such as sterile solutions, suspensions or lyophilized products in the appropriate unit dosage form. Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, CremophorEM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyethylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol or sodium chloride in the composition.

Prolonged absorption of the injectable compositions can be brought about by including in the composition an agent which delays absorption, for example, aluminium monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In a particular embodiment, said pharmaceutical composition is administered via intravenous (IV) or subcutaneous (SC). Adequate excipients can be used, such as bulking agents, buffering agents or surfactants. The mentioned formulations will be prepared using standard methods for preparing parenterally administrable compositions as are well known in the art and described in more detail in various sources, including, for example, “Remington: The Science and Practice of Pharmacy” (Ed. Allen, L. V. 22nd edition, 2012, www.pharmpress.com).

It is especially advantageous to formulate the pharmaceutical compositions, namely parenteral compositions, in dosage unit form for ease administration and uniformity of dosage. Dosage unit form as used herein refers to physically discrete units suited as unitary dosages for the subject to be treated; each unit containing a predetermined quantity of active compound (antibody of the invention) calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Generally an effective administered amount of an antibody of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.001 to 1,000 mg/kg body weight/day, preferably about 0.01 to about 100 mg/kg body weight/day, most preferably from about 0.05 to 10 mg/kg body weight/day. More specifically, for use in accordance with the invention, the anti-CCR7 antibodies are preferably administered at a dosage of 1-1000, 2-500, 5-200, 10-100, 20-50 or 25-35 mg/kg body weight/day, preferably administered in doses every 1, 2, 4, 7, 14 or 28 days.

Aside from administration of antibodies to the patient, the present application contemplates administration of antibodies by gene therapy. WO 96/07321 relates the use of gene therapy to generate intracellular antibodies.

The pharmaceutical compositions can be included in a container, pack, or dispenser together with instructions for administration.

The antibodies and pharmaceutical compositions of this invention may be used with other drugs to provide a combination therapy. The other drugs may form part of the same composition, or be provided as a separate composition for administration at the same time or at different time.

In a further aspect, the invention pertains to an ex vivo or in vitro method for preparing an organ, tissue or cell preparation from a donor for transplantation into a recipient. The method preferably comprises the step of: a) incubating the organ, tissue or cell preparation with an anti-CCR7 antibody or antigen-binding fragment thereof as herein defined, whereby preferably the anti-CCR7 antibody at least one of: i) reduces the number of, and ii) inhibits the activity of, CCR7 expressing donor cells in the organ, tissue or cell preparation; and, b) optionally, removal of at least one of the anti-CCR7 antibody and the CCR7 expressing donor cells from the organ, tissue or cell preparation. Preferably, the anti-CCR7 antibody is incubated with the donor organ, tissue or cell preparation in an amount and for a time that is sufficient/effective to reduce the number of and/or to inhibit the activity of the CCR7 expressing donor cells in the organ, tissue or cell preparation to a degree that is sufficient to reduce the risk of occurrence of GHVD and/or to reduce the severity of GHVD in the recipient of the organ, tissue or cell preparation. For example, the anti-CCR7 antibody is incubated with the donor organ, tissue or cell preparation in an amount and for a time that is sufficient/effective to substantially inhibit the activity of the CCR7 expressing donor cells in the transplant, preferably by at least 40% reduction in activity, more preferably by at least 80% reduction in activity, and most preferably by at least 90% reduction in activity. Or for example, the anti-CCR7 antibody is incubated with the donor organ, tissue or cell preparation in an amount and for a time that is sufficient/effective to substantially decrease the number of CCR7 expressing donor cells in the transplant, preferably by at least 40% reduction in number, more preferably by at least 80% reduction in number, and most preferably by at least 90% reduction in number. It is hereby understood that CCR7 expressing donor cells in the donor organ, tissue or cell preparation preferably are CCR7 expressing immune cells, more preferably including at least one or more of T-lymphocytes, B-lymphocytes, NK cells or APCs.

The method of the invention for preparing an organ, tissue or cell preparation from a donor for transplantation into a recipient preferably is a method that is practiced in an in vitro or ex vivo environment, whereby ex vivo does not exclude that the donor organ, tissue or cell preparation is treated with an anti-CCR7 antibody while still in the body of a brain dead donor, or donor who is dead via circulatory death, by administration of the anti-CCR7 antibody to the donor's body.

All of the above disclosures regarding clinical treatment or prevention of GVHD that is relevant to an in vitro or ex vivo environment applies to this practice. Thus, the anti-CCR7 antibody can be comprised in a preservation solution that is used to preserve the organ, tissue or cell preparation prior to transplantation. For example, the anti-CCR7 antibody may be added to a preservation solution for an organ transplant in an amount sufficient to bind and inhibit activity of immune cells of the organ. In addition, the anti-CCR7 antibody may be added to a preservation solution for an organ transplant in an amount sufficient to bind and decrease the number of immune cells of the organ. Such a preservation solution may be suitable for preservation of different kind of organs such as heart, kidney and liver as well as tissue therefrom. An example of commercially available preservation solutions is Plegisol (Abbott), and other preservation solutions named in respect of its origins include the UW-solution (University of Wisconsin), the Stanford solution and the Modified Collins solution (J. Heart Transplant (1988) Vol. 7(6):456 4467). The preservation solution may also contain conventional co-solvents, excipients, stabilizing agents and/or buffering agents. The preservation solution or buffer containing an anti-CCR7 antibody may also be used to wash or rinse an organ transplant prior to transplantation or storage. Thus, an organ or tissue to be transplanted can be perfused with a preservation solution comprising the anti-CCR7 antibody, preferably prior to transplantation. For example, a preservation solution containing anti-CCR7 antibody may be used to flush perfuse an isolated heart which is then stored at 4° C. in the preservation solution.

In another embodiment, practice of the invention might be used to condition organ or tissue transplants prior to transplantation. Prior to transplantation the anti-CCR7 antibody or fragment may be added to the washing buffer to rid the transplant of active T-lymphocytes, B-lymphocytes, NK cells or APCs.

The concentration of the anti-CCR7 antibody, or fragment, in the preservation solution or wash buffer may vary according to the type of transplant. According to the invention said incubating may e.g. be carried out for from 1 minute to 7 days. As to the removing of at least one of the anti-CCR7 antibody (e.g. unbound anti-CCR7 antibody) and the CCR7 expressing donor cells from the organ, tissue or cell preparation in accordance with the methods and uses of the invention, various ways of performing said step are known to the skilled person. One exemplary way of removing antibody from the graft is by washing the graft. Washing may e.g. occur by employing centrifugation where the graft comprises or is a cell suspension. Alternatively, the anti-CCR7 antibody and the CCR7 expressing donor cells can be removed from a cell preparation to be transplanted (e.g. bone marrow cells, peripheral blood cells, or cord blood cells) by affinity purification of the anti-CCR7 antibody and preferably the CCR7 expressing donor cells bound thereto. Therefore, preferably the affinity ligand used for purification does not affect the antigen binding capacity of the anti-CCR7 antibody such that CCR7 expressing donor cells to the anti-CCR7 antibody can be co-purified from the cell preparation. Methods for affinity purification are well known in the art and include e.g. methods wherein the affinity-ligand is immobilized on solid phase carrier material such as a magnetic bead or a solid phase carrier material as used in affinity (column) chromatography.

The amount of antibody employed in the above step of incubating is not particularly limited. Appropriate amounts may easily be determined by the person skilled in the art and may e.g. depend on the type of graft used. Preferably according to the invention, said incubating is carried out with an antibody amount of from 0.1 μg to 100 mg. The selection of suitable amounts of antibody is well within the expertise of the skilled person. Generally, higher amounts or concentrations, respectively, of antibody are preferred where the graft comprises or is a tissue or an organ. Moreover, the selection of an exact amount or a concentration, respectively, of antibody used will also depend on the size of such tissue or organ.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value (of 10) more or less 0.1% of the value.

All patent and literature references cited in the present specification are hereby incorporated by reference in their entirety.

The present invention is further described by the following examples, which should not be construed as limiting the scope of the invention.

### DESCRIPTION OF THE FIGURES

FIG. 1. Anti-CCR7 antibody is effective in preventing GVHD development.

A) Relative weight loss in the three experimental groups. Control group where mice were treated with PBS (n=4), isotype control (IC) group where mice where treated with an irrelevant antibody (n=5), and anti-CCR7 group where mice were treated with an antibody targeting CCR7 (n=5). Weight at day 0 was considered as 100%. P values refer to comparative analyses of anti-CCR7 group and the other groups.

B) Kaplan-Meier survival curves for all the experimental groups.

C) Percentage of human CD45+ cells found in consecutive samples of peripheral blood (PB) obtained from each experimental group.

D) Percentage of human CD45+ cells found in lymphoid tissues (bone marrow and spleen) collected when animals were euthanized.

FIG. 2. Anti-CCR7 antibody is effective in treating GVHD at early stages.

A) Relative weight loss in the experimental arms. Isotype control (IC) group, where mice were treated with an irrelevant antibody (n=5), and anti-CCR7 group, where mice were treated with an antibody targeting CCR7 (n=5). Weight at day 0 was considered as 100%. P value refers to comparative analyses of anti-CCR7 group and the other group.

B) Kaplan-Meier survival curves for each experimental group.

C) Percentage of human CD45+ cells found in consecutive samples of peripheral blood (PB) obtained from each experimental group.

D) Percentage of human CD45+ cells found in lymphoid tissues (bone marrow and spleen) collected when animals were euthanized.

FIG. 3. Anti-CCR7 antibody is effective in treating GVHD at early and late stages.

A) Relative weight loss in the experimental arms. Isotype control (IC) group where mice were first treated with an irrelevant antibody at day +3 (n=2), at day +7 (n=2), or at day +10 (n=1). Anti-CCR7 groups where mice were first treated with an antibody targeting CCR7 (n=5), at day +7 (n=5), or at day +10 (n=5). Weight at day 0 was considered as 100%. P value refers to comparative analyses of anti-CCR7 group and the other group.

B) Kaplan-Meier survival curves for each experimental group. All animals from each IC arm were grouped in one single group.

FIG. 4. Selection of anti-CCR7 mAb. Several commercial antibody clones targeting CCR7 were characterized based on their ability to block CCR7-mediated migration towards CCL19 and CCL21 (A), and on potency inducing target cell killing mediated by complement (CDC) (B). Both migration (% of input, n=2 in basal, CK, 150503, and 2H4; n=1 in 6B3, 3D12, H60) and CDC (% specific lysis, n=2) were tested on CCR7-expressing chronic lymphocytic leukemia cells following the procedures described in the material and methods section. Bars represent mean±SD. Based on these results, clone 150503 was selected to perform in vitro and in vivo proves of concept in GVHD.

FIG. 5. Mechanisms of action of a neutralizing anti-CCR7 antibody.

A) Blocking CCR7 neutralizes target-mediated cell migration of T_{N} and T_{CM} cells from apheresis. The specific antagonism on CCR7-ligand interactions, expressed as the reduction of % of migrating input cells, is shown for CD4⁺ and CD8⁺ T-cell subsets. In both cases, serum-starved PBMC isolated from apheresis (n=3) were pre-incubated with 10 μg/ml of anti-CCR7 or the respective isotype control (IC) for 30 minutes. Then, chemotaxis induced by 1 μg/ml CCL19 or CCL21 was assayed in nude Transwell chambers (4 hours). Basal migration represents spontaneous migration, without chemotactic stimulus. Cells migrated to the lower chamber were stained and counted by flow cytometry. The percentage of migrated cells (% input) was calculated as stated in Material and Methods.

B) Anti-CCR7 mAb specifically depletes T_{N} and T_{CM}. The specific depletion on CCR7-positive cells, expressed as the % of specific lysis mediated by complement activation (CDC), is shown for CD4⁺ and CD8⁺ T-cell subsets. In both cases, target cells from apheresis (n=3) were incubated with 10 μg/ml of anti-CCR7 or the respective isotype control (IC) for 30 minutes and then exposed to rabbit complement for 1.5 h. Cell lysis was determined through quantification of 7-AAD incorporation in each subset by flow cytometry. The percentage of specific lysis was calculated according to the formula shown in Material and Methods. Bars represent mean±SD. ns, not significant; *, p<0.05; **, p<0.01; *** p<0.001.

FIG. 6. Proportion of infused CCR7+ T-cells subpopulations in the apheresis does not correlate with CMV or relapse rates.

A-B) Proportion of infused CCR7⁺ T-cells subpopulations in the apheresis comparing CMV infection status of the recipients within the first six months after transplantation. Apheresis samples were analyzed by flow cytometry and were divided between those infused into patients who show CMV DNA (n=60) and the ones who do not after the transplant (n=43). The percentage of CD4⁺CCR7⁺ (A) and CD8⁺CCR7⁺ (B) subpopulations infused into patients with or without CMV is shown. To determine CMV reactivation a cut-off value of viral load >57 copies/ml was used.

C-D) Proportion of infused CCR7⁺ T-cells subpopulations in the apheresis comparing patients with or without relapsed disease. Apheresis samples were analyzed by flow cytometry and were divided between those infused into patients who relapsed (n=25) and the ones who did not after the transplant (n=78). The percentage of CD4⁺CCR7⁺ (C) and CD8⁺CCR7⁺ (D) subpopulations infused into patients with or without relapsed disease is shown.

FIG. 7. Proportion of infused CCR7+ T-cells subpopulations in the apheresis does not correlate with relapsing disease. Apheresis samples were analyzed by flow cytometry and were divided between those infused into patients who relapsed (YES) and the ones who did not after the transplant (NO).

The proportion of CCR7⁺ T-cells (CD4⁺ or CD8⁺) subpopulations in the apheresis comparing patients with or without relapsed disease is shown for different blood disorders including:

A) myelodysplastic syndrome (MDS); “YES” ns CD4⁺ p=0.4199; CD8⁺ p=0.2117;

B) acute lymphoblastic leukemia (ALL); “YES” ns CD4⁺ p=0.5758; CD8⁺ p=0.1908;

C) acute myeloid leukemia (AML); “YES” ns CD4⁺ p=0.1638; CD8⁺ p=0.4126;

D) Hodgkin's lymphoma (HD); “YES” ns CD4⁺ p=0.5106; CD8⁺ p=0.8873;

E) non-Hodgkin's lymphoma (NHL); “YES” ns CD4⁺ p=0.9926; CD8⁺ p=0.7369;

F) multiple myeloma (MM).

### EXAMPLES

### Example 1: Antibodies to CCR7 as a Tool for Treating GVHD

### Material and Methods

### Samples, Reagents and Flow Cytometry (FCM)

Peripheral blood samples from healthy volunteers were obtained after informed consent. Analysis of CCR7 expression was subsequently performed on normal T and B lymphocytes. Phycoerythrin (PE)-conjugated mouse anti-human CCR7 was purchased from R&D Systems (McKinley Place, Minn.). In all cases appropriate isotype controls (IC) were included. Immunofluorescence staining was analyzed on a FACS CANTO II flow cytometer using DIVA software (BD Biosciences). Peripheral blood mononuclear cells (PBMC) were isolated by ficoll gradient centrifugation (Histopaque-1077, Sigma-Aldrich, Madrid, Spain).

### Xenogeneic Mouse Model of GVHD

GVHD in vivo models were developed in NOD/SCID-IL2Rγ^{null} mice. To this end, in all models animals were sub-lethally irradiated with 2 Gy, and 4 hours later, 8×10⁶ human peripheral blood mononuclear cells (PBMC) from healthy volunteers (in 200 μl of PBS) were intravenously inoculated into each irradiated mouse. Both 6 to 10 weeks-aged male and female mice were used for the in vivo proof of concept. Experiments were carried out at the animal facilities of Centro de Biología Molecular Severo Ochoa (CBMSO) in accordance with Spanish law and the CBMSO ethic board guidelines.

Clinical parameters evaluated in mice included weight loss, stooped posture (kyphosis), skin alterations, hind leg paralysis (or reduced motility), and tachypnoea. To study infiltration in peripheral blood (PB), blood samples were collected at different times along the experiments. To analyze infiltration in different tissues, mice were euthanized and organs/tissues including spleen and bone marrow (BM) were collected and disaggregated. In both cases, cells were labeled with human-specific anti-CD45 FITC-mAb (Clone H130, BD Biosciences, www.bdbiosciences.com), and then analyzed by flow cytometry.

### Preventive Use of Anti-CCR7 Antibody in Mice

To evaluate the efficacy of blocking CCR7 in donor cells, mice were used in preventive settings. To this end, mice were first treated with either a purified murine anti-human-CCR7 mAb (n=5 mice; clone 150503, isotype IgG2a, R&D Systems, Minneapolis, Minn., USA) or an irrelevant isotype control (IC) antibody (n=5 mice; IgG2a, Biolegend, San Diego, Calif., USA) or PBS (n=4 mice). Both anti-CCR7 mAb and IC were intra-peritoneally injected at ˜10 mg/kg (˜200 μg/mouse). After 2 hours, each animal was inoculated with PBMCs from a single healthy donor. Animals received 4 more doses of anti-CCR7, IC or PBS every 4 days. PB samples were analyzed on days 10, +13, +18 y +21 post-transplant. BM and Spleen were analyzed after euthanizing the animals.

### Therapeutic Use of Anti-CCR7 Antibody During GVHD Peaks

To study the therapeutic efficacy of anti-CCR7 mAb, a model was developed in order to evaluate whether anti-CCR7 mAb impacted on allo-reactivity populations found in PB, and whether this approach attenuated GVHD symptoms. In this model PBMC-bearing mice were first treated at day +5 post-engraftment with anti-CCR7 (n=5) or with its corresponding IC (n=5) at ˜10 mg/kg (˜200 μg/mouse). Treatment was repeated every 3 days. PB sampling was carried out on days +10, +13, +18 y +25 post-transplantation. Spleen and BM sampling was carried out when animals were euthanized. Experiment was terminated 33 days after PBMCs engraftment.

By means of another model, we evaluated efficacy of using anti-CCR7 in different time points during or after the allo-reactivity peak. To this end, twenty human PBMC-bearing mice where treated with anti-CCR7 (n=15) or an IC (n=5). Within the anti-CCR7 treated group, 5 mice received first dose at day +3; 5 mice received first dose at day +7; and 5 mice received first dose at day +10. Within IC treated group, 2 mice were first treated on day +3; 2 mice on day +7 and one mouse on day +10 after engraftment. Experiment was terminated on day +26.

### Assay for Determining Inhibition of CCR7-Dependent Intracellular Signalling

The ability of an anti-CCR7 antibody to inhibit the CCL19- and/or CCL21-mediated intracellular signalling in human CCR7 overexpressing Chinese Hamster Ovary (CHO) cells, was determined by an established standard β-arrestin recruitment assay (PathHunter™, DiscoverX, Fremont, Calif., USA; Southern et al., 2013, J Biomol Screen. 18(5):599-609).

### Assay for Determining Inhibition of Cell Migration

The ability of an anti-CCR7 antibody to inhibit the migration (chemotaxis) of human T cell lymphoma cells, endogenously expressing the human CCR7 receptor, induced by ligands CCL19 and CCL21, was determined in cell migration assays.

Cell migration assays were performed using transwell double chambers with inserts of 8 μm pore size (Costar, Cambridge, Mass., USA). The lower chamber contained the ligand (CCL19 or CCL21) diluted in HamF12 medium supplemented with 0.5% BSA. The CCR7 endogenous expressing cells (T-cell lymphoma (HuT-78)), pre-incubated with anti CCR7 monoclonal antibodies, were placed into the insert and the chamber assembly was incubated at 37° C. The amount of transmembrane migrated cells in the lower chamber was determined, after cell lysis, by DNA staining (CyQuant GR dye solution, Life Technologies Ltd, UK).

### Assay for Complement-Dependent Cytotoxicity (CDC)

CDC assay was performed as described in Cuesta-Mateos et al (Cancer Immunol Immunother. 2015, 64: 665-76). Briefly, 2×10⁵ PBMC target cells were plated in a 96-well round-bottom plate together with the indicated concentrations of purified anti-CCR7, alemtuzumab (anti-CD52) or IC antibodies. After 30 min at 37° C., the cells were washed and complete RPMI 1640 medium containing 25% rabbit complement (Serotec, Oxford, UK) with or without prior heat inactivation (56° C., 30 min) was added. After 1.5 h, the cells were stained with anti-CD19-FITC, anti-CD3-PE and anti-CD5-APC mAb to discriminate between CLL cells and T cell populations. 7-AAD was used as a viability exclusion dye. The percentage of specific lysis (% SL) was calculated with the formula: 100×(% dead cells with activated complement−% dead cells with inactivated complement)/(100−% dead cells with inactivated complement).

### Assay for Determining Absence of Agonistic Effects

Tested at high concentrations (267 nM), anti-human CCR7 binding monoclonal antibodies were tested for (absence of) induced detectable intracellular agonistic effects in human CCR7 overexpressing Chinese Hamster Ovary (CHO) cells, using an established standard β-arrestin recruitment assay (PathHunter™, DiscoverX, Fremont, Calif., USA; Southern et al., 2013, J Biomol Screen. 18(5):599-609) (data not shown). An unrelated IgG2a was used as negative control, and CCL21, a natural ligand for CCR7, was used as positive control. An anti-human CCR7 binding antibody is found to lack detectable intracellular agonistic effects if the antibody induces no more intracellular agonistic effects than the negative control.

### Biacore Affinity Measurement

The affinities of the monoclonal antibodies were determined by Biacore measurements under standard conditions. The monoclonal antibody was immobilized on an appropriate sensor surface and the solution of the sulfated antigen SYM1899 ((pyroGlu)DEVTDDZIGDNTTVDZTLFESLCSKKDVRNK; SEQ ID NO: 3); wherein Z denotes sulfated Tyrosine) comprising residues 19-49 derived from the N-terminus of human CCR7, was passed over the sensor surface.

### Results

### Preventive Administration of Anti-CCR7 Blocks GVHD Development

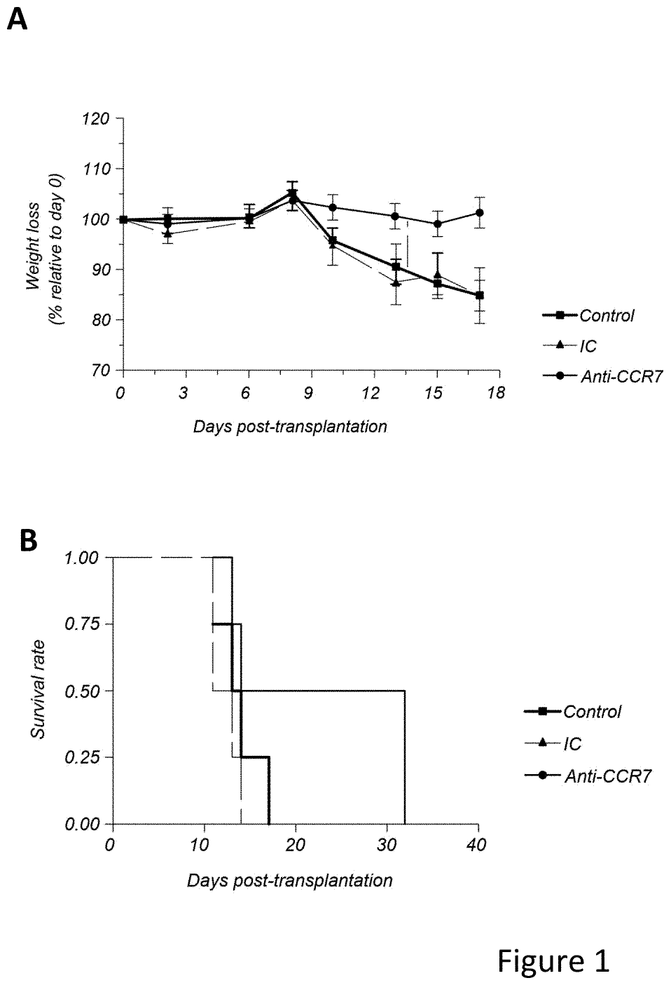

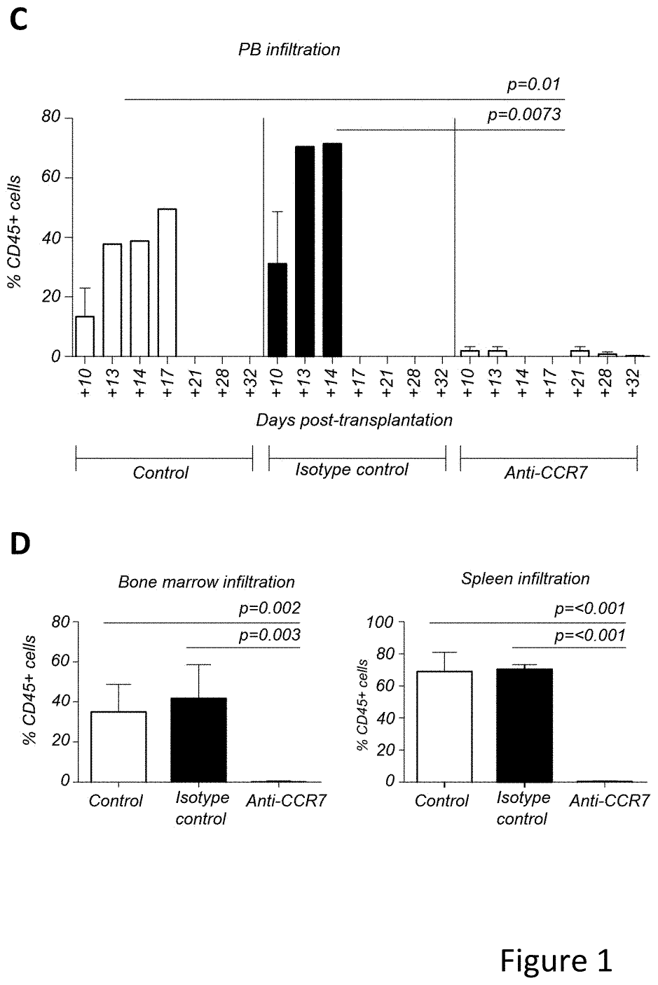

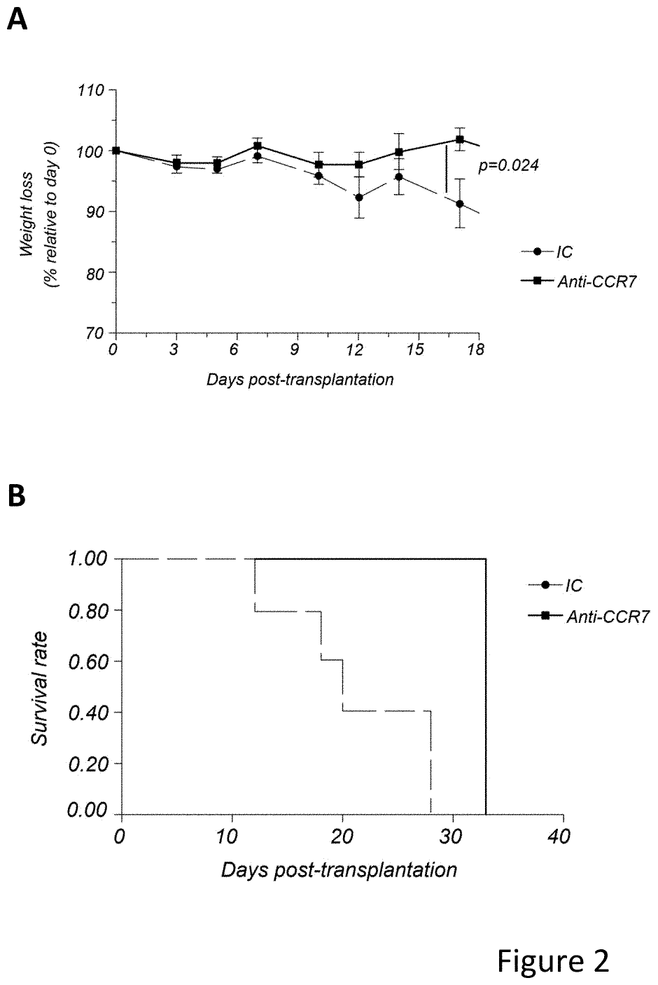

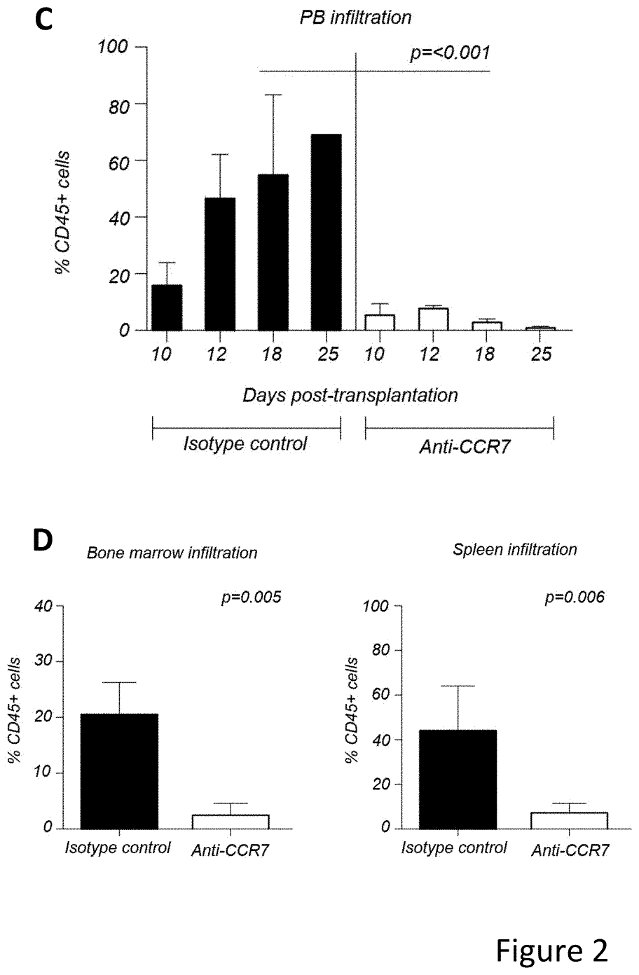

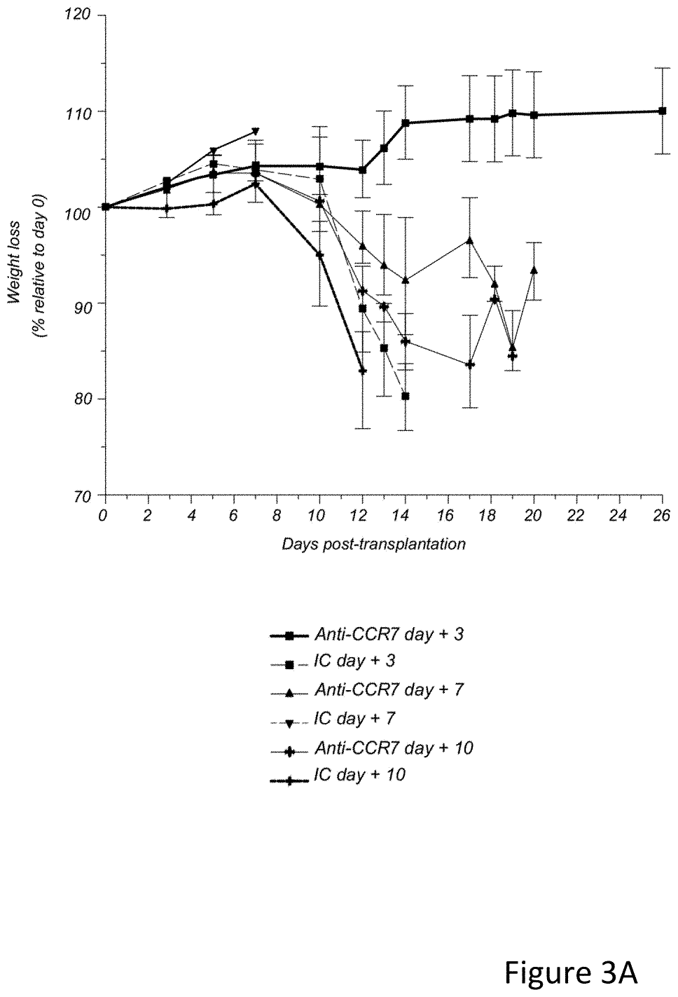

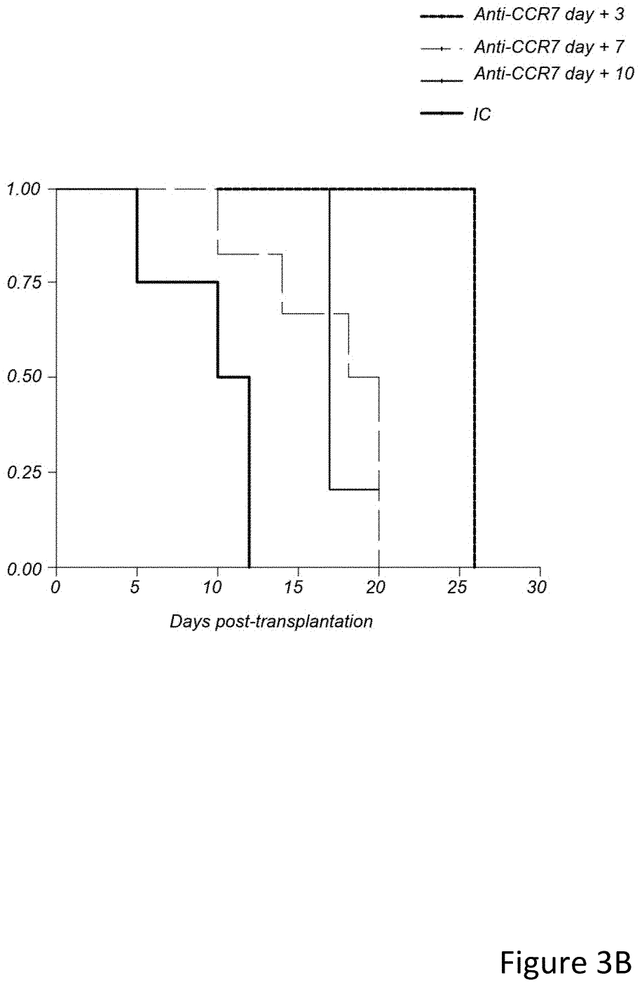

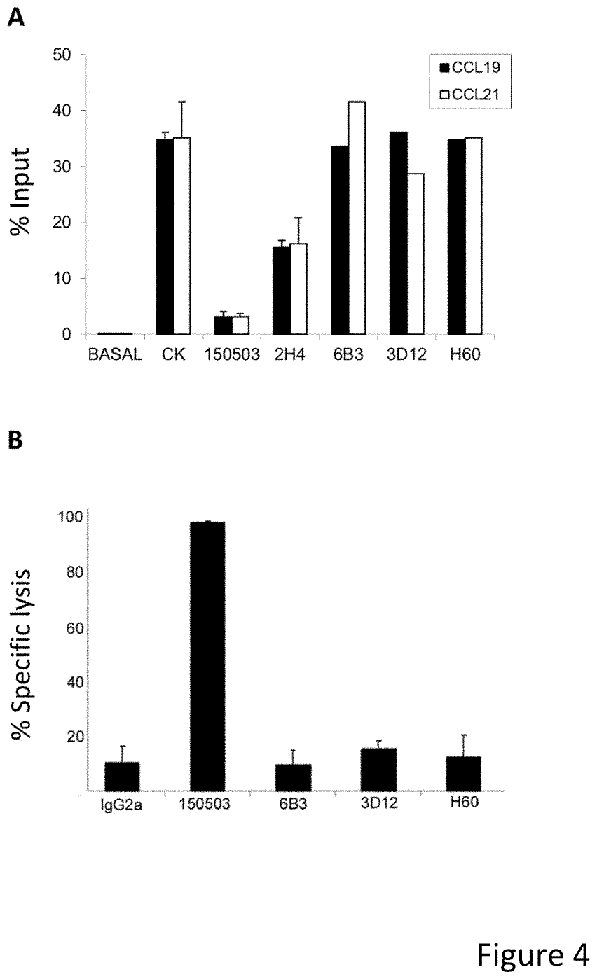

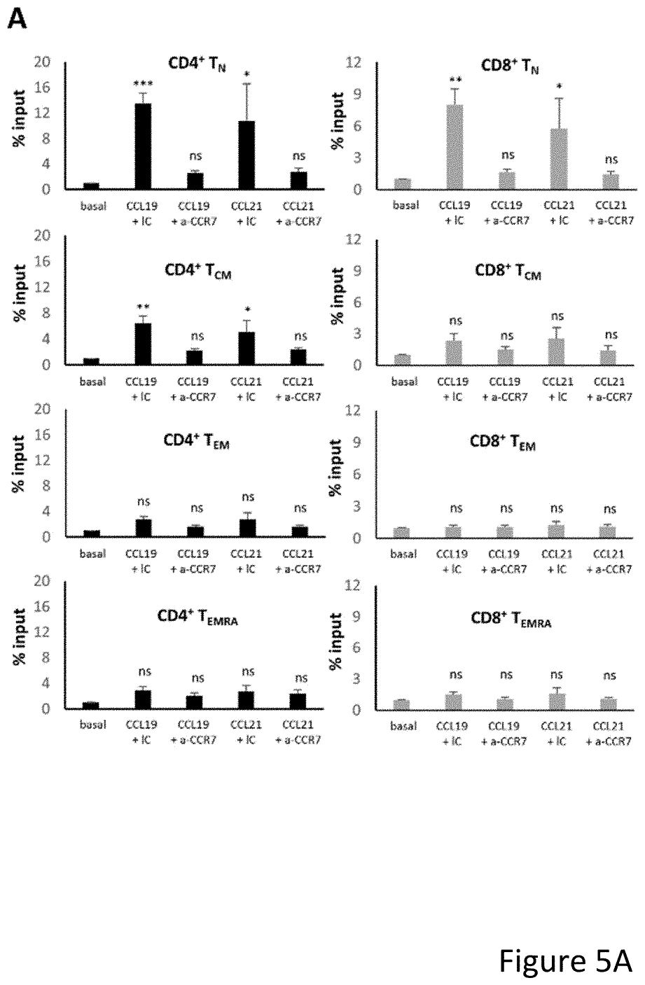

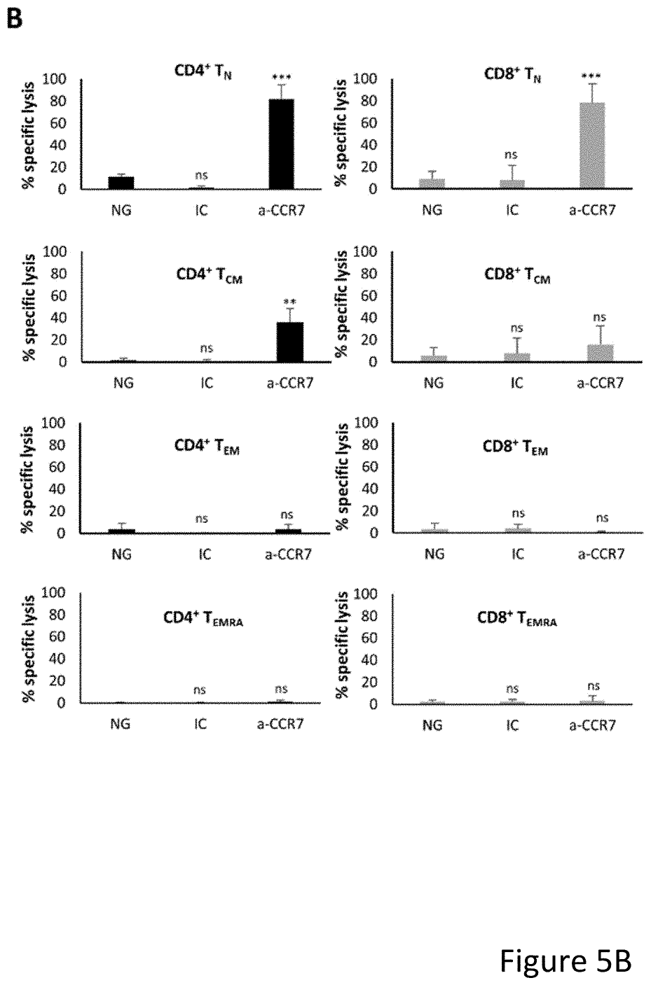

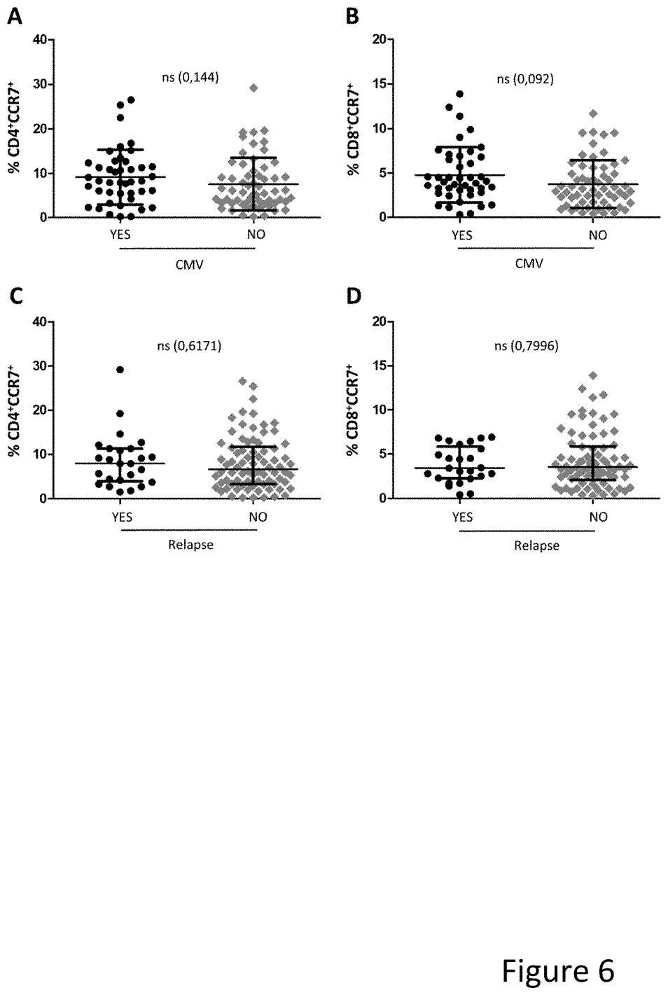

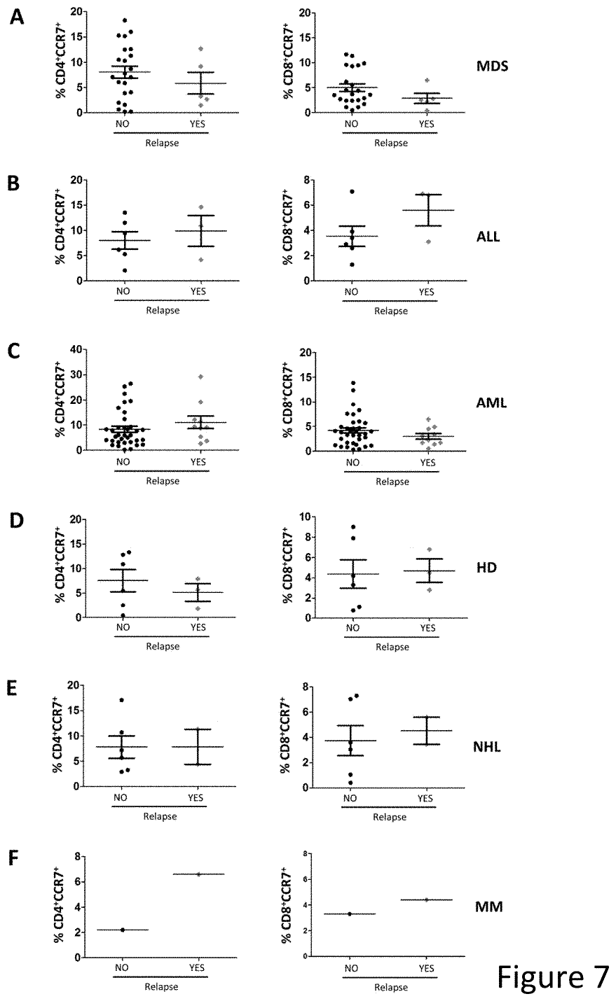

Mice receiving a first preventive dose of anti-CCR7 prior to engraftment of hPBMCs, and four consecutive doses after engraftment, did not developed any GVHD clinical sign. In contrast, mice receiving IC or PBS showed clinical signs, including weight loss (FIG. 1A). Weight differences were observed between days +9 and +12 post-transplantation (IC vs anti-CCR7, p=0.045; and PBS vs anti-CCR7, p=0.0134). Notably, animals receiving anti-CCR7 even gained weight all over the experiment. Accordingly, anti-CCR7 antibody extended overall survival (FIG. 1B). Animals treated with the anti-CCR7 mAb did not develop any clinical sign and survived up to 32 days, the time point when animals were sacrificed and which was considered as a bona fide disease-free period. In contrast, control mice presenting severe clinical signs were euthanized on days +11, +13, +14, and +18. On days +13, +14 and +18, one animal from anti-CCR7 treated grouped was sacrificed in order to arrange comparative analyses on organ infiltration. None of the animals receiving anti-CCR7 antibody showed clinical signs and they were sacrifice for purely experimental purposes. Accordingly, during these days, the anti-CCR7 treated mice did not develop any clinical sign and gained weight and, accordingly, no presence of reactive donor cells was detected in PB from anti-CCR7-treated mice (FIG. 10). In contrast, the presence of pro-GVHD cells in PB of controls increased over the time. At the time of sacrifice, infiltration was analyzed in BM and spleen (FIG. 1D). In line with findings in PB, no pro-GVHD cells were seen in lymphoid tissues from animals treated with anti-CCR7 mAb. Conversely, there was a constant infiltration of these tissues in the control group (BM Control vs BM anti-CCR7: 34.6% vs 0.57%, p=0.002; BM IC vs BM anti-CCR7: 41.7% vs 0.57%, p=0.003/Spleen Control vs spleen anti-CCR7: 70.1% vs 0.17%, p=<0.001; spleen IC vs spleen anti-CCR7 71.3% vs 0.17%, p=<0.001). No differences were observed between control groups (PBS vs IC: BM, p=0.57/spleen, p=0.86).

### Therapeutic Administration of Anti-CCR7 Ameliorates GVHD

To demonstrate the therapeutic efficacy of anti-CCR7 antibodies in vivo, we used models wherein animals were treated once allo-reactive responses were developed. These responses, which used to take place on days +3 to +5, are the main cause in the GVHD pathogenicity. That said, in one model human PBMCs were engrafted into immune-deficient mice. Animals were treated with either an IC (n=5) or an anti-CCR7 antibody (n=5). The first dose of the antibodies was administrated on day +5 and consecutive dosing was done every two days. In this model, anti-CCR7 antibody positively impacted on weight of animals (FIG. 2A). In contrast, control animals lost weight. Differences were first observed by day +12. In addition, anti-CCR7 therapy extended overall survival (FIG. 2B).

Animals treated with the anti-CCR7 mAb did not develop any clinical sign and survived up to 33 days, the time point when the animals were sacrificed and which was considered as a bona fide disease-free period. In contrast, control mice presenting severe clinical signs were euthanized on days +12, +20, and +28. On days +12, and +28, one animal from anti-CCR7 treated group was sacrificed in order to arrange comparative analyses on organ infiltration. None of these animals receiving anti-CCR7 antibody showed clinical signs and sacrifice had experimental purposes. Accordingly, during these days, anti-CCR7 treated mice did not develop any clinical sign and gained weight and, accordingly, no presence of reactive donor cells was detected in PB from anti-CCR7-treated mice (FIG. 2C). In contrast, the presence of pro-GVHD cells in PB of controls increased over the time. Notably, significant differences in PB infiltration were observed from day +10 (control group vs anti-CCR7 group:12.6% vs 2.6%; p=0.02). These differences increased in day +12 and keep different until the end of the experiment (47.3% vs 6.5%; p<0.001) (FIG. 2C). At the time of the sacrifice, infiltration was analyzed in BM and spleen (FIG. 2D). In line with findings in PB, a small proportion of pro-GVHD cells were seen in lymphoid tissues (BM and spleen) from animals treated with anti-CCR7 mAb (FIG. 2D). Conversely, there was a constant infiltration of these tissues in the control group (BM Control vs BM anti-CCR7: 27.3% vs 3.5%; p=0.005/Spleen Control vs spleen anti-CCR7: 59.2% vs 8.4%; p=0.006).

In another model, we aimed to evaluate the efficacy of anti-CCR7 antibodies in treating GVHD at different times after the onset of disease. To this end, anti-CCR7 antibodies were administrated in different time points after engraftment. Animals were treated on days +3, +7, and +10 after engraftment of donor PBMCs. Fifteen mice were treated with anti-CCR7 antibody (5 on day +3; 5 on day +7; 5 on day +10) and five mice were treated with an IC (2 on day +3; 2 on day +7; and 1 on day +10). All mice received consecutive doses every two days until the end of the experiment. Mice receiving their first dose of anti-CCR7 antibody on day +3 showed gain of weight and an extended overall survival (FIGS. 3A and 3B). Animals treated with the anti-CCR7 mAb did not develop any clinical sign and survived up to 26 days, time when were sacrificed as was considered as a bona fide disease-free period. In contrast, control mice showed a median overall survival of 14 days. Notably, animals treated with the anti-CCR7 antibody not before day +7 or +10, showed worst outcome than mice wherein the treatment started on day +3. However, the animals where the treatment started only at day +7 or +10 still showed a better outcome than their respective controls. Some animals receiving first dose on days +7 or +10 lived until day +19 whereas no animal in the respective control groups survived longer than day +12.

Anti-CCR7 Antibody Impairs Human T_{N} and T_{CM} cells in vitro chemotaxis towards CCL19 and CCL21

These results prompted us to evaluate the utility of CCR7 not as a biomarker to select the proper graft but as a targetable receptor for antibody-based therapy. To do that, we selected and used an antibody featured by the ability to block CCR7-ligands interactions and killing target cells through CDC or antibody-dependent cellular cytotoxicity (ADCC) (FIG. 4).

Then, we first evaluated the ability of the selected mAb to inhibit ligands-driven chemotaxis of hPBMC from apheresis. As expected, when PBMC were preincubated with an IC, the addition of CCL19 or CCL21 to the medium triggered migration of CCR7⁺ T_{N} and T_{CM} subsets (FIG. 5A), and had the more prominent effect in the T_{N} compartment. However, the binding of 10 μg/ml anti-CCR7 mAb reduced migration to basal levels in these cells. Conversely, T_{EM} and T_{EMRA} did not migrate in response to CCR7 ligands and, accordingly, anti-CCR7 did not impact their behavior.

Anti-CCR7 Antibody Specifically Depletes CCR7⁺ human T_{N} and T_{CM} cells through CDC

As stated before (Cuesta-Mateos C. Targeting CCR7 in T-cell Prolymphocytic Leukemia. CONTROL-T: International Conference April 2016 Mature T-cell lymphomas—molecular pathology, modeling of cellular dynamics, and therapeutic approaches. 2016) the selected antibody was powerful enough to kill tumor T-cells through CDC, but its effect on healthy CCR7⁺ T-cell subsets was not addressed before. We, therefore, performed in vitro CDC assays with fresh hPBMC from apheresis. Upon binding to CD4⁺ T_{N} or T_{CM} cells, 10 μg/ml anti-CCR7 mediated a powerful CDC activity (FIG. 5B). Similar results were observed in CD8⁺ T_{N} cells. Conversely, anti-CCR7 mAb spared CCR7-negative T_{EM} and T_{EMRA}, indicating that anti-CCR7 therapy will maintain effector cells and, consequently, the protection against pathogens and the GVL effect. To explore this idea further, we studied whether the numbers of CCR7⁺ cells in the graft correlated with CMV reactivation within the first 6 months after the transplantation but no clear differences in the proportion (FIGS. 6A and 6B) or absolute numbers (data not shown) of CD4⁺CCR7⁺ or CD8⁺CCR7⁺ cells were seen between patients with or without CMV reactivation. Further, a multivariate logistic regression analysis (Table 1) confirmed that the proportion of CCR7⁺ cells in the graft was not a risk factor for CMV reactivation

TABLE 1
Multivariate analysis

| | OR | p-value^{a} | 95% CI |
|---|---|---|---|
| CMV reactivation (yes/no) | 0.95 | 0.144 | 0.889-1.0173 |
| CD4⁺CCR7⁺ (%) | | | |
| CMV reactivation (yes/no) | 0.88 | 0.092 | 0.772-1.019 |
| CD8⁺CCR7⁺ (%) | | | |
| Relapsed disease (yes/no) | 1.01 | 0.702 | 0.942-1.092 |
| CD4⁺CCR7⁺ (%) | | | |
| Relapsed disease (yes/no) | 0.92 | 0.362 | 0.779-1.095 |
| CD8⁺CCR7⁺ (%) | | | |

Abbreviations:
CMV, cytomegalovirus;
CI, confidence interval;
OR, odds ratio;
^{a}Adjusted by the significant confounding variables.

[CD4⁺ (p=0.144); CD8⁺ (p=0.092)]. Similarly, the proportion or absolute numbers of CCR7⁺ cells in the graft did not correlated with rates of relapsed disease after the transplantation and, again, no clear differences were seen between patients who relapsed and those who not (FIGS. 6C and 6D and data not shown). Accordingly, a multivariate logistic regression analysis (Table 1) confirmed that the proportion of CCR7⁺ cells in the graft was not a risk factor for relapsed disease [CD4⁺ (p=0.702); CD8+(p=0.362)]. Finally, the lack of association between proportion of CCR7⁺ cells in the graft and relapse incidence was further confirmed when patients were grouped according to the diagnosis of the underlying disease (FIG. 7). All together, these results precluded the use of CCR7 (in the apheresis) as a biomarker to predict CMV infection or relapsed disease, but as an add-on read out it suggested that any approach aiming to reduce the proportion of CCR7⁺ cells in the graft would not be associated with a higher risk of infection or a higher rate of relapses.
Anti-CCR7 mAb Block CCR7 Signalling with No Agonistic Effects

Tested at high concentrations (267 nM), a monoclonal antibody having the HVRs of SEQ ID NO.'s 1 and 2 did not show any detectable agonistic effect in human CCR7 overexpressing Chinese Hamster Ovary (CHO) cells as determined by an established standard B-arrestin recruitment assay (PathHunter™, DiscoverX, Fremont, Calif., USA; Southern et al, 2013, J Biomol Screen. 18(5):599-609) (data not shown).

### Discussion

GVHD is a frequent complication derived after allogenic transplantation that may be fatal. Has been recently demonstrated that higher CCR7 expression in donor cells correlates with higher grade of recipient secondary lymphoid organs (SLOs) infiltration, thus with a higher chance to find allo-antigens which may lead to allogenic immune responses. Previous data from inventors demonstrated that migration to SLOs relies on CCR7 and associations between migration towards CCR7 ligands and the development and grade of GVHD has been established (Portero-Sainz, I et al., Bone Marrow Transplantation (2017), 1-8). Similarly, other publications propose that naïve T cell and TCM are the main players in the development of both aGVHD and cGVHD (Yakoub-Agha, I., et al., Leukemia, 2006. 20(9): p. 1557-65; Distler, E., et al., Haematologica, 2011. 96(7): p. 1024-32; Cherel, M., et al., Eur J Haematol, 2014. 92(6): p. 491-6.), although naïve T-cells have a greater ability to respond against recipient antigens than TCM. These data suggest the possibility of using CCR7 as therapeutic target in immunotherapy not only due to their high density in naïve T-cells TCM, and several APCs, but also because of its crucial role in disease progression and pathogenicity. In this sense, we demonstrated in vivo that administration of anti-CCR7 antibodies to NHP led to a selective reduction of naïve T cells, as well as TCM cells (data not shown). Moreover, anti-CCR7 antibodies have shown to be effective in blocking migration of CCR7-expressing T-cells towards CCR7 ligands (data not shown). Finally, anti-CCR7 antibodies are effective in preventing and treating GVHD as demonstrated with in vivo mouse models. Notably, the most efficacy therapeutic approach involved administration of anti-CCR7 antibodies on days +3 to +5, thus mirroring the therapeutic window in which cyclophosphamide is used to prevent allo-reactivity in HSCT (Luznik, L., et al., Biol Blood Marrow Transplant, 2002. 8(3): p. 131-8). Altogether, results on preclinical use of anti-CCR7 antibodies confirm that depleting and/or neutralizing migration of CCR7-expressing cells (including naïve T-cells and TCM) to SLOs are rational approaches to prevent and/or to treat GVHD. Therefore, by depleting CCR7-expressing cells, and/or by blocking migration to SLOs, allo-reactive CCR7-expressing cells will not be activated, thus impairing development of GVHD.

Accordingly, Sasaki et al. (2003, J Immunol, 170(1): p. 588-96.) showed that early use of CCL21 antagonists prevented entry of donor T-cells into the lymph node and thus GVHD development. Dutt et al. showed that depletion of naïve CD62L cells delayed GVHD onset and extended OS in pre-clinical in vivo models (Dutt, S., et al., Blood, 2005.106(12): p. 4009-15). Similarly, in clinical settings, depletion of CD45RA-expressing naïve T cells from apheresis impacted on incidence and development of GVHD (Touzot, F., et al., J Allergy Clin Immunol, 2015.135(5): p. 1303-9 e1-3; Shook, D. R., et al., Pediatr Blood Cancer, 2015. 62(4): p. 666-73; Triplett, B. M., et al., Bone Marrow Transplant, 2015. 50(7): p. 1012.). However, in all these works, and in contrast to anti-CCR7 therapy, TCM cells are not targeted. To conclude, is worth to mention that recent evidence suggests that immunity against infections is not depending on CCR7+ cells (Choufi, B., et al., Bone Marrow Transplant, 2014. 49(5): p. 611-5.) thus depleting and/or blocking CCR7-expressing cells seems to be a safety approach for recipient patients.

### Example 2: Identification of Patients Having Low Risk of GVHD

### Material and Methods

We analyzed a cohort of 103 donor-recipient pairs (see Table 2) who underwent allo-HSCT at the La Princesa University Hospital, Madrid, Spain (Portero-Sainz et al, 2017). The study protocols were approved by the Ethics Committee (Reference PI-624) and performed in accordance with the Declaration of Helsinki.

TABLE 2
Transplant characteristics

| Transplant characteristics | no. of patients (%) | |
|---|---|---|
| Recipients age, years | | |
| 0-20 | 2 | (2%) |
| 21-30 | 13 | (13%) |
| 31-40 | 21 | (20%) |
| 41-50 | 26 | (25%) |
| 51-60 | 25 | (25%) |
| >60 | 16 | (15%) |
| Diagnosis of the underlying | | |
| disease [Relapse rate] | | |
| Myelodisplastic syndrome (MS) | 28 (27%) | [5/28 (17%)] |
| Acute lymphoid leukemiac (ALL) | 9 (9%) | [3/9 (33%)] |
| Acute myeloid leukemia (AML) | 45 (44%) | [10/45 (22%)] |
| Hodgkin lymphoma (HL) | 9 (9%) | [3/9 (33%)] |
| Non-Hodgkin Lymphoma (NHL) | 8 (8%) | [2/8 (25%)] |
| Chronic lymphocytic leukemia (CLL) | 2 (2%) | [0/2 (0%)] |
| Multiple myeloma (MM) | 2 (2%) | [1/2 (50%)] |
| Donors age, years | | |
| 17-30 | 33 | (32%) |
| 31-40 | 32 | (31%) |
| 41-50 | 19 | (19%) |
| 51-60 | 13 | (12%) |
| >60 | 6 | (6%) |
| Gender matching | | |
| D male - R male | 36 | (35%) |
| D male - R female | 26 | (25%) |
| D female - R female | 18 | (18%) |
| D female - R male | 23 | (22%) |
| Donor type | | |
| HLA-identical sibling | 31 | (30%) |
| HLA-identical (10/10) unrelated | 47 | (46%) |
| HLA- Cw mismatched (9/10) | 25 | (24%) |
| unrelated | | |
| CMV serology (I) | | |
| D positive - R negative | 12 | (12%) |
| D positive - R positive/D negative - | 77 | (75%) |
| R positive | | |
| D negative - R negative | 14 | (13%) |
| CMV serology (II) | | |
| Reactivation | 59 | (57%) |
| Source of graft | | |
| BMSC | 5 | (5%) |
| PBSC | 98 | (95%) |
| Infused CD34⁺_10⁶/kg of recipient | 5.23 | (1.4-8.2) |
| weight | | |
| Infused CD3⁺_10⁶/kg of recipient | 22.07 | (25.3-468.1) |
| weight | | |
| Conditioning regimen | | |
| Myeloablative | 70 | (68%) |
| No Myeloablative Standard | 33 | (32%) |
| GVHD prophylaxis | | |
| Cs A and MTX | 90 | (87%) |
| Cs A and MMF | 11 | (11%) |
| Cs A | 2 | (2%) |

Abbreviations: D, donor; R, recipient; CMV, cytomegalovirus; BMSC, bone marrow stem cells; PBSC, peripheral blood stem cells; CsA, cyclophosphamide; MTX, methotrexate; MMF, mycophenolate mofetil.

### Phenotyping

The apheresis samples were stained with a seven-color panel of antibodies (Table 3) as previously described (Portero-Sainz et al, 2017). Relative and absolute numbers of the T-cell subsets refer to the total white blood cell counts. T_{N}, T_{CM}, T_{EM}, and T_{EMRA} subsets were identified with the following antibodies: CD45RA-FITC, CD62L-PE, CD3-APC, CD4-PB (BD Biosciences, San Jose, Calif.).

TABLE 3
Antibody clones used in the study.

| Target | Clone | Fluorochrome | Source |
|---|---|---|---|
| CD8 | SK1 | FITC | BD |
| CD4 | RPA-T4 | PB | BD |
| CD62L | SK11 | PE | BD |
| CD3 | SK7 | PerCP | BD |
| CD3 | SK7 | APC | BD |
| CD19 | SJ25C1 | PECy7 | BD |
| CD45 | 2D1 | PO | BD |
| CD45RA | HI100 | FITC | BD |
| CCR7 | 150503 | APC | R&D |
| CCR7 | 150503 | None (purified) | R&D |
| IgG2a | MOPC-173 | None (purified) | BIOLEGEND |

### Therapeutic Antibodies

Purified mouse anti-human CCR7 mAb (IgG2a isotype) was purchased from R&D Systems (MN, USA), and the matched isotype control (IC) from Biolegend (CA, USA).

### Statistical Analysis

Qualitative variables are presented as relative (percentage, %) and absolute (number, n) frequencies. Quantitative variables are expressed as measures of central tendency (mean) and dispersion (SD or SEM). Qualitative data between groups were compared by Pearson's χ2 test or Fisher exact test, as appropriate. Quantitative variables with equal variances (Levene's test) were analyzed using the t test or one-way analysis of variance (ANOVA). Mann-Whitney U or Kruskal-Wallis tests were used for heterocedasticity.

Binary logistic regression analysis were performed as appropriate in the cohort of patients described by Portero-Sainz et al (2017) to identify predictors of GVHD and CMV reactivation (or relapsed disease) adjusted by confounding variables: age, HLA and CMV status, sex, conditioning regimen, number of CD3⁺ and CD34⁺ infused, allo-sensitization, relapse postHSCT, base disease, source of graft and prophylaxis.

An exploratory univariate analysis was performed to search for variables associated with the dependent variables aGVHD, cGVHD, and CMV infection or relapsed disease (P<0.05). Confounding variables reaching a probability threshold on univariate analysis (P<0.10) were included in a multivariate logistic regression model. Sensitivity ±95% CI from the aGVHD risk score from our previous model (Portero-Sainz et al, 2017) was estimated by means of ROC curves. Sensitivity from CMV infection risk score was calculated in the same way. Significance was set at a value of P<0.05. To determine CMV reactivation a cut-off value of viral load >57 copies/ml was used. Statistical analysis was performed using Stata version 13.0 (College Station, Tex., USA).

### Results

Apheresis Selection Based on the Proportion of CCR7⁺ Cells does not Prevent or Delay GVHD

To establish a potential cut-off point to select for those apheresis with a low risk of developing GVHD we performed sensitivity analysis (ROC curves) on our cohort and arbitrarily selected the 25^{th} percentile (<3.6%) to identify patients transplanted with a low proportion of CCR7+ T cells. As seen in Table 4, 87.88% (75.23%-100%) patients receiving grafts with a proportion of CD4⁺CCR7⁺ cells within the 25^{th} percentile (<3.6%) did not develop aGVHD. In the case of CD8⁺CCR7⁺, the selection of <2.2% cells in the apheresis (25th percentile) was associated with a sensitivity of 88.57% (76.60%-100%) for cGVHD.

TABLE 4
ROC analysis of % CD4+CCR7+ (or % CD8+CCR7+) cells
in the apheresis and aGVDH (or cGVHD) (with a cut-off
set on 25th percentile)
aGVHD (no patients)

| % CD4+CCR7+ | no | yes | Total |
|---|---|---|---|
| <3.6 | 21 | 4 | 25 |
| >=3.6 | 49 | 29 | 78 |
| Total | 70 | 33 | 103 |

| % CD4+CCR7+ | value | CI (95%) |
|---|---|---|
| Sensitivity (%) | 87.88 | 72.23-100 |
| Specificity (%) | 30.00 | 18.55-41.45 |
| TPR | 37.18 | 25.81-48.55 |
| TNR | 84.00 | 67.63-100 |
| Prevalence | 32.04 | 22.54-41.54 |

cGVHD (no patients)

| % CD8+CCR7+ | no | yes | Total |
|---|---|---|---|
| <2.2 | 18 | 4 | 22 |
| >=2.2 | 35 | 31 | 66 |
| Total | 53 | 35 | 88 |

| % CD8+CCR7+ | value | CI (95%) |
|---|---|---|
| Sensitivity (%) | 88.57 | 76.60-100 |
| Specificity (%) | 33.96 | 20.27-47.66 |
| TPR | 46.97 | 34.17-59.77 |
| TNR | 81.82 | 63.43-100 |
| Prevalence | 39.77 | 28.98-50.57 |

1.-15. (canceled)

16. A method for preventing or treating Graft Versus Host Disease (GVHD) in a recipient of a transplant comprising a donor cell, the method comprising administering to the recipient an anti-CCR7 antibody or antigen-binding fragment thereof.

17. The method according to claim 16, wherein the anti-CCR7 antibody or antigen-binding fragment thereof has an IC50 of no more than 100 nM for inhibiting at least one of CCR7-dependent intracellular signaling and CCR7 receptor internalization, by at least one CCR7-ligand selected from CCL19 and CCL21.

18. The method according to claim 17, wherein the anti-CCR7 antibody or antigen-binding fragment thereof inhibits CCR7-dependent intracellular signaling without substantial agonistic effects.

19. The method according to claim 16, wherein the anti-CCR7 antibody or antigen-binding fragment thereof has a Kd for the N-terminal extracellular domain of human CCR7 that is not more than a factor 20 higher than the Kd of a reference anti-CCR7 antibody, whereby the reference anti-CCR7 antibody is a mouse anti-CCR7 antibody of which the amino acid sequence of the heavy chain variable domain is SEQ ID NO: 1 and of which the amino acid sequence of the light chain variable domain is SEQ ID NO: 2.

20. The method according to claim 16, wherein the anti-CCR7 antibody thereof is a chimeric, humanized or human antibody.

21. The method according to claim 16, wherein the anti-CCR7 antibody is an antibody having the HVRs of the anti-human CCR7 antibody of which the amino acid sequence of the heavy chain variable domain is SEQ ID NO: 1 and of which the amino acid sequence of the light chain variable domain is SEQ ID NO: 2.

22. The method according to claim 21, wherein the anti-CCR7 antibody effects at least one of killing, inducing apoptosis, blocking migration, blocking activation, blocking proliferation and blocking dissemination of CCR7 expressing cells in the recipient.

23. The method according to claim 21, wherein the transplant comprising the donor cell is a transplant comprising one or more of an organ, tissue, a progenitor cell, a stem cell and a hematopoietic cell.

24. The method according to claim 23, wherein the transplant comprising the donor cell is a transplant comprising a hematopoietic stem or progenitor cell.

25. The method according to claim 24, wherein the recipient suffers from a malignant disorder.

26. The method according to claim 25, and wherein the prevention or treatment of GHVD maintains or promotes the graft versus tumor effect or the graft versus leukemia effect.

27. The method according to claim 16, wherein the prevention or treatment of GHVD comprises at least one of:
a) administrating the anti-CCR7 antibody or antigen-binding fragment thereof to the recipient prior to that the recipient receives the transplant comprising the donor cell;
b) administrating the anti-CCR7 antibody or antigen-binding fragment thereof to the recipient after that the recipient has received the transplant comprising the donor cell, and preferably before that the recipient shows symptoms of GHVD or before that the recipient has been diagnosed with GHVD;
c) administrating the anti-CCR7 antibody or antigen-binding fragment thereof to the recipient after that the recipient has received the transplant comprising the donor cell, and preferably after that the recipient shows symptoms of GHVD or after that the recipient has been diagnosed with GHVD;
d) administrating the anti-CCR7 antibody or antigen-binding fragment thereof to the recipient of a transplant comprising the donor cell, which transplant has been prepared prior to transplantation by an ex vivo incubation with the anti-CCR7 antibody or antigen-binding fragment thereof; and,
e) administrating the anti-CCR7 antibody or antigen-binding fragment thereof to the recipient after recurrence of GHVD.

28. An ex vivo method for preparing an organ, tissue or cell preparation from a donor for transplantation into a recipient, the method comprising the steps of:
a) incubating the organ, tissue or cell preparation with an anti-CCR7 antibody or antigen-binding fragment thereof as defined in claim 17, whereby the anti-CCR7 antibody effects at least one of:
i) a reduction of the number of, and ii) an inhibition of the activity of, CCR7 expressing donor cells in the organ, tissue or cell preparation; and,
b) optionally, removal of at least one of the anti-CCR7 antibody and the CCR7 expressing donor cells from the organ, tissue or cell preparation.

29. The method according to claim 28, wherein the anti-CCR7 antibody is comprised in a preservation solution used to preserve the organ, tissue or cell preparation prior to transplantation.

30. The method according to claim 29, wherein the organ or tissue is perfused or washed with the preservation solution comprising the anti-CCR7 antibody.

31. The method according to claim 28, wherein the anti-CCR7 antibody and the CCR7 expressing donor cells are removed from the cell preparation by affinity purification of the anti-CCR7 antibody and the CCR7 expressing donor cells bound thereto.


SEQUENCE LISTING (10 sequences)

SEQ ID NO: 1 - protein, 125 aa, Mus musculus
EVQLVESGGDLVKPGGSLKLSCVVPGLTFRDFAMSWVRQIPEKRLQWVATISSGGFYTYYPDSVKGRFTISRDNAKNTLYLQMNSLRSEDTAMYYCVRRAYRYDGTGDYSALDYWGQGTSVTVSS

SEQ ID NO: 2 - protein, 107 aa, Mus musculus
DIQMTQSPSSLSASLGERVSLTCRASQDIGPSLNWLQQGPDGTIKRLIYATSNLDSGVPKRFSGSRSGSDYSLTISSLESEDFVDYYCLQFASSPLTFGAGTKLELK

SEQ ID NO: 3 - protein, 32 aa, Artificial
  SYM1899 synthetic sulfated peptide corresponding to amino acids 19-49 of human CCR7
  MOD_RES (1)..(1): PYRROLIDONE CARBOXYLIC ACID
  MOD_RES (8)..(8): SULFATATION
  MOD_RES (17)..(17): SULFATATION
EDEVTDDYIGDNTTVDYTLFESLCSKKDVRNK

SEQ ID NO: 4 - protein, 125 aa, Artificial
  Humanized VH1 domain
EVQLLESGGGFVKPGGSLKLSCVVSGLTFRDFAMSWVRQTPEKRLVWVATISSGGFYTYYPDSVKGRFTISRDNVRNILYLQMSSLRSEDTAVYYCVRRAYRYDGTGDYSALDYWGTGTTVTVSS

SEQ ID NO: 5 - protein, 107 aa, Artificial
  Humanized VK1 domain
DIQMTQSPSSLSASVGDRVTITCRASQDIGPSLNWYQQKPGKAPKRLIYATSNLDSGVPSRFSGSGSGTDFTLTISSLQPEDFATYYCLQFASSPLTFGQGTKVEIK

SEQ ID NO: 6 - protein, 107 aa, Artificial Sequence
  Synthetic human antibody
SYELTQPPSVSVSPGQRARITCSGDKLGDKYASWYQQKPGQSPLLVIYQDSKRPSGIPERFSGSNSGNTATLTISGTQAMDEADYYCQAWGSSTVIFGGGTKLTVLG

SEQ ID NO: 7 - protein, 106 aa, Artificial Sequence
  Synthetic human antibody
YELTQPPSVSVSPGQRARITCSGDKLGDKYASWYQQKPGQSPLLVIYQDSKRPSGIPERFSGSNSGNTATLTISGTQAMDEADYYCQAWGSSTVIFGGGTKLTVLG

SEQ ID NO: 8 - protein, 124 aa, Artificial Sequence
  Synthetic human antibody
QVQLVESGGGVVQPGRSLRLSCAASGFTFSSYGMHWVRQAPGKGLEWVAVIWYDGSKKYYADSVKGRFTISRDNSKNTLYLQMNRLRAEDTAVYYCARDGGIAVFLQDNWFDPWGQGTLVTVSS

SEQ ID NO: 9 - protein, 330 aa, Homo sapiens [sequence omitted]

SEQ ID NO: 10 - protein, 330 aa, Artificial
  synthetic heavy chain constant region of the human allotype G1m (17,1) with a E333A substitution
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIAKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK